(19) United States
(12) Patent Application Publication
(10) Pub. No.: US 2003/0099962 A1
(43) Pub. Date: May 29, 2003

(54) Methods to isolate gene coding and flanking DNA

(75) Inventors: Johann Schernthaner, Orleans (CA); Caroline Piche, Aylmer (CA); Laurian Robert, Gatineau (CA)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Her Majesty the Queen in Right of CA as Rep. by the Minister of Agriculture and Agri-Food

(21) Appl. No.: 10/093,365

(22) Filed: Mar. 8, 2002

Related U.S. Application Data
(60) Provisional application No. 60/274,239, filed on Mar. 9, 2001.

(51) Int. Cl. C12Q 1/68; C12P 19/34; C12N 15/00

(52) U.S. Cl. 435/6; 435/91.2; 435/320.1

(57) ABSTRACT

The invention provides methods for obtaining gene coding fragments, flanking fragments, or both gene coding and flanking fragments that can be readily characterized and used for a variety of purposes. The method involves preparing a genomic library comprising genomic DNA fragments, and hybridizing one or more nucleic acid primers, selected from a full-length cDNA, a 5′ cDNA end, a 3′ cDNA end, or a combination thereof, or a full-length mRNA, or portion thereof, or an RNA fragment, or a combination thereof, to the population of single stranded DNAs. A second strand is synthesized using a nucleic acid polymerase and the hybridized one or more nucleic acid primers, and a double stranded nucleic acid is produced. Any single stranded nucleic acid is removed and the nucleic acid reconstituted to produce a vector. This method is suitable for high throughput preparation and analysis of gene coding regions or flanking regions that can be used for preparing DNA arrays. Therefore, the present invention also provides arrays comprising flanking fragments, or flanking fragments attached to coding fragments. The present invention also pertains to promoter sequence tags, and 3′ sequence tags.

The present invention relates to methods of isolating DNA sequences. More specifically, this invention relates to methods of isolating and analyzing coding and flanking regions of genes.

### BACKGROUND OF THE INVENTION

Recent developments in molecular biology have brought about high throughput gene sequencing and analysis. The large scale sequencing of cDNA libraries is yielding an ever-increasing number of partial gene sequences (Expressed Sequence Tags, ESTs). There are now more than 10 million EST sequences in the GenBank public database. Similarly, new technologies like DNA microarrays now allow the monitoring of gene expression of thousands of genes simultaneously. These new genomics tools are having a tremendous impact on biological research. The present invention provides methods to generate and analyze large numbers of genes and associated regulatory sequences.

EST sequencing provides a high throughput, genome-size-independent method of accessing coding regions within genomes. However, there is at present no high throughput method that can isolate 5′ and 3′ gene regulatory sequences within complex genomes. One method that can be used to obtain regulatory regions on a large scale is genome sequencing. However, genome sequencing on a large scale is not only expensive, but it is time intensive as is accurately identifying the regulatory regions involved in gene expression within genome sequences. Accordingly, the number of characterized regulatory regions has not kept pace with the number of cDNA sequences in public databases.

Regulatory sequences are essential to regulate gene expression, yet their function remains poorly understood and largely unpredictable. Furthermore, there exist no methods to identify or evaluate large numbers of gene regulatory regions simultaneously. Promoters for example, are typically isolated from genomic DNA clones and analyzed one at a time.

Genome sequencing is inaccessible to most individual laboratories and is presently suitable for only a limited number of organisms. Many important crops such as wheat and maize, for example, have genomes that are exceedingly large due to the occurrence of large tracts of non-coding DNA. Much of this DNA needs to be sequenced in order to identify DNA representative of structural genes and their associated regulatory sequences. This problem is exacerbated in polyploid organisms, for example alfalfa, wheat, canola, and oat. Similar challenges exist in the analysis and characterization of animal genomes.

Typically, promoter sequences have been obtained by screening genomic libraries with known nucleic acid sequences such as cDNAs and identifying the portion of the DNA upstream from the coding region. This approach is quite reliable in yielding regulatory and genomic sequences of the gene of interest, however it is labour intensive, normally requires the screening of large numbers of genomic clones typically using one probe at a time, and requires significant post-screening manipulation and analysis. Therefore, this approach makes screening of genomic libraries difficult to scale up.

Methods to isolate 3′ or 5′ ends of individual cDNAs are well established (e.g. Frohman, M. A., et al., Proc. Natl. Acad. Sci. USA, 1988, 85:8998-9002). Similarly, methods to isolate gene flanking sequences of individual genes using the polymerase chain reaction (PCR) have been developed (Hui, E. K.-W., et al., Cell. Mol. Life Sci., 1998, 54:1403-1411; U.S. Pat. Nos. 5,470,722; 4,994,370). However, these methods require the prior knowledge of part of the gene sequence to enable the isolation of additional sequences of the gene. This makes scaling up difficult since genes need to be obtained individually or in small numbers.

Enrichment for promoters and 5′ transcribed regions from large genomic clones has also been demonstrated by enriching for DNA with Sp-1 binding sites (Mortlock, D. P., et al., Genome Res., 1996, 6:327-335). In this approach the transcription factor binding the target site in the promoter is used to carry out the enrichment for an Sp-1 containing regulatory region. However, this method cannot be applied to every gene, and is only suitable for identifying the family of regulatory regions comprising Sp-1 sites.

The use of constructs which trap or tag genes and regulatory sequences within the genome of various organisms have been described (for example see Cecconi, F. and Meyer, B. I., FEBS Letters, 2000,480:63-71; Springer, P., Plant Cell, 2000,12:1007-1020). However, the approach of insertional mutagenesis is not practical for many organisms and generally lies outside the scope of individual laboratories, since it requires screening and maintaining large populations of living organisms. Furthermore, this method is biased to regions of DNA that are readily tagged or trapped. This method also requires significant effort to sort out individually the genetics of the insertion(s), and the exact nature of the DNA sequence(s) that was tagged.

A recent development involves high throughput generation of gene-trap sequence tags (GTSTs) which represent DNA sequences flanking genomic insertions (Parinov, S., et al., Plant Cell, 1999, 11:2263-2270; Wiles, M. V. et al., Nature Genet., 2000, 24:1-14). This method has the potential of yielding large numbers of sequences from genes and their regulatory sequences, however it suffers from many of the drawbacks of insertional mutagenesis mentioned above such as a limited range of suitable organisms, bias to insertion sites, insertion complexity, difficulties to reach saturation, and the requirement for a large scale effort to elucidate the genetics of the insertion events, and analyze the trapped DNAs.

None of the methods mentioned above, are adapted for high throughput identification and functional analysis of thousands of regulatory elements simultaneously. With new developments in DNA macroarray and microarray technologies it is now possible to monitor the expression patterns of thousands of genes in a single experiment (for example see Granjeaud, S. et al., BioEssays, 1999, 21:781-790). For example, thousands of cDNAs can be spotted on a glass slide (microarray) or on filter membrane (macroarray) and studied by hybridization to labelled cDNA probes. These approaches are very efficient at revealing expression profiles of genes, however, they are not well suited for the analysis of regulatory regions. To obtain regulatory sequences responsible for the observed expression pattern, one still needs to resort to the time consuming methods described above, one gene at a time.

In cases where the genome of an organism has been sequenced, it is possible to generate arrays containing flanking sequences by amplifying these sequences using primers deduced from the known sequence. For example, such an array from yeast was screened to identify DNA sequences bound by transcription factors Gal4 and Ste12 (Ren, B., et al., Science, 2000, 290:2306-2309). However, such a process requires prior knowledge of the sequence of an organism's genome.

The present invention bypasses many of the difficulties identified in the prior art by enriching for gene sequences and their associated regulatory regions. The present method does not require prior knowledge of gene sequence and allows high throughput isolation of gene coding and flanking regions. Furthermore, the present invention permits the high throughput sequencing of flanking sequences (Flanking Sequence Tags, FSTs) or promoter sequences (Promoter Sequence Tags, PSTs) and of 3′ flanking sequences (3′ Sequence Tags, TSTs) at a scale comparable to ESTs.

The present invention does not require the development, screening and maintenance of large populations of organisms, nor does it involve complicated genetic and molecular analyzes of individually tagged organisms. The present invention is also amenable to large-scale saturation analyzes of genes and associated regulatory regions since it relies on material that is readily available from most organisms, namely, mRNA and genomic DNA.

The methods provided in the present invention bypass difficulties identified in the prior art in isolating and analyzing regulatory regions on a high throughput scale, for example using micro, or macro arrays, by having the flanking fragments directly attached to the coding fragments for which the expression patterns are observed. Therefore, the promoters for any or all genes on the array that display an expression pattern of interest can be obtained directly.

It is an object of the invention to overcome disadvantages of the prior art.

The above object is met by the combinations of features of the main claims, the sub-claims disclose further advantageous embodiments of the invention.

### SUMMARY OF THE INVENTION

The present invention relates to methods of isolating DNA sequences. More specifically, this invention relates to methods of isolating coding and flanking regions of genes.

According to the present invention there is provided a method (A) to obtain a vector comprising one or more gene coding fragments, flanking fragments, or a combination thereof, comprising:

i) providing a population of single stranded DNAs from a genomic library;

ii) hybridizing one or more nucleic acid primers to the population of single stranded DNAs;

iii) synthesizing a second strand from the single stranded DNAs using a nucleic acid polymerase, and the hybridized one or more nucleic acid primers, and producing a double stranded nucleic acid;

iv) removing single stranded nucleic acid; and

v) reconstituting the vector.

The present invention pertains to the above method (A), wherein in the step of hybridizing (step iii)), the one or more nucleic acid primer is a full-length cDNA, a 5′ cDNA end, a 3′ cDNA end, or a combination thereof, or a full-length mRNA, or portion thereof, or an RNA fragment, or a combination thereof.

The present invention also embraces a method (B) to obtain a vector comprising one or more gene coding fragments, flanking fragments, or a combination thereof, comprising:

i) providing a population of single stranded DNAs from a genomic library;

ii) hybridizing one or more nucleic acid primers to the population of single stranded DNAs, the one or more nucleic acid primers selected from the group consisting of 5′ cDNA ends, 3′ cDNA ends, RNA ends, and a combination thereof,

iii) synthesizing a second strand from the single stranded DNAs using a nucleic acid polymerase, and the hybridized one or more nucleic acid primers, and producing a double stranded nucleic acid;

iv) removing single stranded nucleic acid; and

v) reconstituting the vector.

The present invention also provides for a method (C) to obtain a vector comprising genomic DNA enriched for coding fragments, flanking fragments, or a combination thereof, comprising:

i) providing a population of single stranded genomic DNA fragments;

ii) hybridizing one or more nucleic acids to the population of single stranded genomic DNA fragments;

iii) synthesizing a second strand from the single stranded genomic DNA fragments using a nucleic acid polymerase, and the hybridized one or more nucleic acids, and producing double stranded nucleic acid;

iv) removing single stranded nucleic acid, to produce a population of double stranded DNA; and

v) introducing the population of double stranded DNA into the vector; and

vi) reconstituting the vector.

The present invention pertains to the above method (C), wherein in the step of hybridizing (step ii)), the one or more nucleic acid primer is a full-length cDNA, a 5′ cDNA end, a 3′ cDNA end, or a combination thereof, or a full-length mRNA, or portion thereof, or an RNA fragment, or a combination thereof.

The present invention pertains to a method (D) to obtain a vector comprising genomic DNA enriched for flanking fragments comprising:

i) providing a population of single stranded DNAs comprising genomic DNA fragments from a genomic library;

ii) hybridizing a modified first nucleic acid, and one or more second nucleic acids to the population of single stranded DNA;

iii) synthesizing a second strand from the single stranded DNA using a nucleic acid polymerase and the one or more second nucleic acids as a primer for nucleic acid synthesis, and producing double stranded nucleic acid;

iv) removing single stranded nucleic acid; and

v) reconstituting the vector.

The present invention pertains to the above method (D), wherein in the step of hybridizing (step iii)), the one or more second nucleic acid primer is a full-length cDNA, a 5′ cDNA end, a 3′ cDNA end, or a combination thereof, or a full-length mRNA, or portion thereof, or an RNA fragment, or a combination thereof. Furthermore, the present invention embraces the above method (D), wherein in the step of hybridizing (step iii)), the modified first nucleic acid comprises an amine or thiol group at its 3′ end.

The present invention also provides a method (E) to obtain a vector comprising genomic gene coding regions enriched for flanking fragments comprising:

i) providing a population of single stranded DNAs comprising genomic DNA fragments from a genomic library;

ii) hybridizing a first nucleic acid to the population of single stranded DNAs and linearizing the vector, to produce a population of linearized vectors;

iii) hybridizing one or more second nucleic acids to the population of linearized vectors;

iv) synthesizing a second strand from the linearized plasmids using a nucleic acid polymerase and the one or more second nucleic acids as a primer for nucleic acid synthesis, and producing a double stranded nucleic acid;

v) removing single stranded nucleic acid; and

vi) reconstituting the vector.

The present invention pertains to the above method (E), wherein, in the step of hybridizing (step iii)), the one or more nucleic acid primer is a full-length cDNA, a 5′ cDNA end, a 3′ cDNA end, or a combination thereof, or a full-length mRNA, or portion thereof, or an RNA fragment, or a combination thereof. Furthermore, the present invention embraces the above method (E), wherein in the step of hybridizing (step iii)), the modified first nucleic acid comprises an amine or thiol group at its 3′ end.

The present invention also pertains to a method of preparing an array comprising a plurality of flanking fragments with attached coding fragments, comprising, obtaining a population of reconstituted vectors produced by any one of methods (A), (B), (C), (D) or (E) defined above, and applying the population of reconstituted vectors, their inserts or parts thereof onto a matrix.

Also the present invention includes a method to produce cDNA ends comprising,

i) providing RNA;

ii) hybridizing cDNA fragments to mRNA to produce a DNA/RNA hybrid;

iii) producing DNA/RNA fragments from the DNA/RNA hybrid;

iv) selecting the DNA/RNA fragments that comprise the cDNA end; and

v) removing the RNA.

Preferably, the mRNA is full-length mRNA.

The present invention also relates to an alternate method of producing cDNA ends comprising,

i) providing DNA fragments;

ii) obtaining cDNA within a vector;

iii) hybridising cDNA to DNA fragments to produce a DNA/DNA hybrid, or a portion thereof;

iv) generating DNA/DNA fragments from the DNA/DNA hybrid;

v) selecting the DNA/DNA fragments that comprise the cDNA end; and

vi) recovering one member of the DNA/DNA fragment.

Preferably, the cDNA is full-length cDNA.

The present invention also provides:

a promoter sequence tag (PST), or a PST produced by any one of methods (A), (B), (C), (D) and (E), where the one or more nucleic acid primers is a cDNA end, or an RNA end. Preferably, the cDNA end is a 5′ cDNA end;

a 3′ sequence tag (TST), or a TST produced by any one of methods (A), (B), (C), (D) and (E), where the one or more nucleic acid primers is a cDNA end, mRNA end, or an RNA end. Preferably, the cDNA end is a 3′ cDNA end;

an array comprising a plurality of 5′ flanking fragments, where the 5′ flanking fragments are attached to coding fragments;

an array comprising a plurality of 3′ flanking fragments, where the 3′ flanking fragments are attached to coding fragments;

an array comprising a plurality of 5′ flanking fragments and 3′ flanking fragments, where the 5′ flanking fragments and 3′ flanking fragments are attached to coding fragments;

an array comprising a plurality of 5′ flanking fragments, or a portion thereof;

an array comprising 3′ flanking fragments, or a portion thereof;

a vector comprising a plurality of contiguous dT's adjacent a restriction site that is capable of cleaving the 3′ end of the contiguous dT's;

pMUSC1;

a vector comprising a nucleotide sequence that when digested with an appropriate restriction enzyme produces a 3′ overhang of contiguous dG's;

pMUSC2;

a vector comprising a stem-loop adaptor sequence, the stem-loop adaptor sequence capable of forming a hybridized stem-loop structure when the vector is in single stranded form, the hybridized stem loop structure comprising one or more restriction sites;

pHMSL1: and

pHMSL2.

By using the methods as described herein the difficulties identified associated with isolating and analyzing regulatory regions, flanking regions on a high throughput scale within the prior art are overcome. By providing arrays, that have the flanking fragments attached to coding fragments, for which the expression patterns are observed, 5′, 3′ or both 5′ and 3′ flanking regions for any or all genes on the array which display an expression pattern of interest can be easily obtained.

This summary of the invention does not necessarily describe all necessary features of the invention but that the invention may also reside in a sub-combination of the described features.

### BRIEF DESCRIPTION OF THE DRAWINGS

These and other features of the invention will become more apparent from the following description in which reference is made to the appended drawings wherein:

FIG. 1 shows a schematic representation of an aspect of an embodiment of the present invention for the enrichment of genomic DNA for gene coding and flanking sequences using a population of nucleic acids, such as cDNAs (FIG. 1(A) and (B)), or mRNAs (FIGS. 1(C) and (D)). FIG. 1(A) shows an embodiment where the population of nucleic acids are cDNAs, and where, for simplicity, there are no introns in the genomic DNA. FIG. 1(B) shows an embodiment where the populations of nucleic acids are cDNAs, and an intron (30) is present within the genomic DNA. The single stranded genomic DNA is shown as a thick line (5), the black sections of the thick line represent flanking regions and introns (when present), the white sections of the thick line correspond to coding sequence. The single stranded genomic DNA is hybridized to cDNAs and second strands are synthesized. The second strand being synthesized is shown as a thin line (50) with an arrowhead indicating the direction of DNA synthesis. Single stranded DNAs are removed and the double stranded plasmids are re-circularized. This approach can enrich for plasmids containing coding sequences, introns, and, 5′ and 3′ flanking DNA. FIG. 1(C) shows an embodiment where the population of nucleic acids are mRNAs. FIG. 1(D) shows an embodiment where the populations of nucleic acids are mRNAs, and an intron is present within the genomic DNA. The single stranded genomic DNA is shown as a thick line with the black sections representing flanking regions and introns (when present) and the white section corresponding to coding sequence. The plasmids containing single stranded genomic DNA are hybridized to mRNAs and once poly (A) tails are removed, second strands are synthesized. The second strand being synthesized is shown as a thin line with an arrowhead indicating the direction of DNA synthesis. The plasmids are re-circularized, the single stranded nucleic acids removed and the double stranded plasmids remain. This approach can enrich for plasmids containing coding sequences, introns, and 5′ and 3′ flanking DNA.

FIG. 2 shows an alternate schematic representation of an aspect of an embodiment of the present invention for the enrichment of genomic DNA comprising gene coding and flanking sequences using genomic DNA as a template. FIG. 2(A) shows the use of linearized genomic DNA as a template for the enrichment of genomic coding, 3′ flanking, or 5′ flanking, regions. The genomic DNA corresponds to linear DNA fragments and is shown as a thick line with the black sections representing flanking regions and the white section corresponding to coding sequence. The genomic DNA is hybridized with a population of nucleic acids tagged with one member of an affinity pair, for example, a biotinylated population of nucleic acids, and the complementary strand is synthesized. The tagged (e.g. biotinylated) DNA is captured using the second member of an affinity pair, for example streptavidin, the single stranded DNA removed, and the remaining double stranded DNA cloned into a suitable vector. FIG. 2(B) shows a schematic representation for the enrichment of genomic DNA for gene coding and flanking sequences using a plasmid-specific oligonucleotide (small arrow (25), the “first nucleic acid”) and a population of nucleic acids (20, “second nucleic acid”). The first nucleic acid may be chemically modified or unmodified at its 3′ end and complementary to the region of the plasmid next to the genomic DNA insert. The single stranded genomic DNA is shown as a thick line with the black sections representing flanking regions and the white section corresponding to coding sequence. The single stranded plasmids containing genomic DNA are first hybridized to the first nucleic acid and the resulting double stranded region is cleaved with a restriction enzyme. The linearized single stranded plasmids containing the genomic DNA are then hybridized with the second nucleic acids and second strands are synthesized. The synthesized second strand is shown as a thin line. Single stranded DNA is removed and the double stranded plasmids are re-circularized. This approach can enrich for plasmids containing coding sequences (partial if introns are present) and 5′ or 3′ flanking DNA. FIG. 2(C) shows a schematic representation for the enrichment of genomic DNA for gene coding and flanking sequences using aplasmid-specific oligonucleotide (small arrow, (25) the “first nucleic acid”) and a population of nucleic acids (20, “second nucleic acid”). The first nucleic acid is preferably chemically modified at its 3′ end and complementary to the region of the plasmid next to the genomic DNA insert. The single stranded genomic DNA is shown as a thick line with the black sections representing intron and flanking regions, and white sections corresponding to coding sequences. The single stranded plasmids containing genomic DNA are hybridized to the first and second nucleic acids and second strands are synthesized. The synthesized second strand is shown as a thin line. Single stranded DNA is removed and the plasmids are re-circularized. This approach can enrich for plasmids containing coding sequences, or partial sequences (if introns are present), and 5′ or 3′ flanking DNA. FIG. 2(D) shows a schematic representation for the enrichment of genomic DNA for gene coding and flanking sequences using a plasmid-specific oligonucleotide (small arrow, the “first nucleic acid”) and a sub-population of cDNAs (for example, enriched for cDNA 5′ ends or 3′ ends; the “second nucleic acid”). The first nucleic acid is preferably chemically modified at its 3′ end and complementary to the region of the plasmid next to the genomic DNA insert. The single stranded genomic DNA is shown as a thick line with the black sections representing intron and flanking regions, and white sections corresponding to coding sequences. The single stranded plasmids containing genomic DNA are hybridized to the first and second nucleic acids and second strands are synthesized. The synthesized second strand is shown as a thin line. Single stranded DNA is removed and the plasmids are re-circularized. This approach can enrich for plasmids containing partial coding sequences and 5′ or 3′ flanking DNA. FIG. 2(E) shows a schematic representation for the enrichment of genomic DNA for gene coding and flanking sequences using a plasmid-specific oligonucleotide (small arrow, the “first nucleic acid”) and a sub-population of cDNAs (for example, enriched for cDNA 5′ ends or 3′ ends; the “second nucleic acid”). The first nucleic acid is preferably chemically modified at its 3′ end and complementary to the region of the plasmid next to the genomic DNA insert. The single stranded genomic DNA is shown as a thick line with the black sections representing intron and flanking regions, and white sections corresponding to coding sequences. In this representation the plasmid contains the sacB gene for levansucrase from Bacillus subtilis (eg. as in vector pHSacX-T1) and it is represented as a grey box. The sacB gene has been inserted into the plasmid region located between the binding site of the plasmid-specific oligonucleotide (small arrow, the “first nucleic acid”) binding site on the plasmid and the cloned genomic DNA insert such that the sacB gene should remain single-stranded and be removed as described above. In situations where the sacB gene is not removed along with the other single-stranded DNA, bacteria containing plasmids with the sacB gene can be selected against by growing them on sucrose. The single stranded plasmids containing genomic DNA are hybridized to the first and second nucleic acids and second strands are synthesized. The synthesized second strand is shown as a thin line. Single stranded DNA is removed and the plasmids are re-circularized. This approach can enrich for plasmids containing partial coding sequences and 5′ or 3′ flanking DNA. FIG. 2(F) shows a schematic representation of the enrichment of genomic DNA for gene coding and flanking sequences using a plasmid-specific oligonucleotide (small arrow, the “first nucleic acid”) and a population of mRNAs (“second nucleic acid”). The first nucleic acid is preferably chemically modified at its 3′ end and complementary to the region of the plasmid next to the genomic DNA insert. The single stranded genomic DNA is shown as a thick line with the black sections representing intron and flanking regions, and white sections corresponding to coding sequences. The single stranded plasmids containing genomic DNA are hybridized to the first and second nucleic acids, and once poly (A) tails and any non-hybridized RNA is removed, second strands are synthesized. The second strand being synthesized is shown as a thin line with an arrowhead indicating the direction of DNA synthesis. Single stranded nucleic acids are removed and the plasmids are re-circularized. This approach can enrich for plasmids containing coding sequences (partial if introns are present) and 3′ flanking DNA.

FIG. 3 shows a schematic representation of the enrichment for a sub-population of antisense cDNA 5′ fragments representing the 5′ ends of mRNAs using mRNA as the initial template. Full-length tagged mRNAs comprising one member of an affinity pair (for example biotin) are hybridized with small cDNA fragments followed by a RNase I treatment. DNA/RNA hybrids are captured using a second member of an affinity pair (for example, streptavidin), RNA removed, and the antisense cDNA 5′ fragments obtained. These single stranded cDNA sub-populations direct synthesis from the coding region towards the 5′ flanking region and thus can be used to isolate 5′ coding and flanking DNA.

FIG. 4 shows a schematic representation of an embodiment of the present invention for the enrichment for a sub-population of antisense cDNA 5′ fragments representing the 5′ ends of mRNAs using cDNA as the initial template for PCR. FIG. 4(A) shows a method for the enrichment for a sub-population of antisense cDNA 5′ fragments using a tagged primer (tagged with one member of an affinity pair, for example, biotin) complementary to a plasmid sequence upstream of the cDNA insert in combination with another primer complementary to the plasmid region downstream of the insert, so that during PCR, the sense strands are tagged with one member of an affinity pair. Following PCR, the double stranded cDNA products are fragmented and the tagged (e.g. biotinylated) DNA/DNA hybrids are captured using the other member of the affinity pair (for example, streptavidin). The antisense cDNA 5′ fragments are then eluted from the bound hybrid or the DNA/DNA hybrids are released from the bound vector portion by restriction digest and are denatured into single strands before use. FIG. 4(B) shows an alternate method for the enrichment for a sub-population of antisense cDNA 5′ fragments using PCR. Sense strands are synthesized from linearized template using a tagged (e.g. biotinylated) primer complementary to the plasmid region upstream of the cDNA insert. The tagged sense strands are hybridized with small cDNA fragments (80) and treated with a DNA nuclease. Tagged DNA/DNA hybrids are captured using the second member of an affinity pair, for example streptavidin. The antisense cDNA 5′ fragments are then eluted from the bound hybrid or the DNA/DNA hybrids are released from the bound vector portion by restriction digest and are denatured into single strands before use. These single stranded cDNA sub-populations direct synthesis from the 5′ end of the coding region towards the 5′ flanking region and thus can be used to isolate 5′ coding and flanking DNA.

FIG. 5 shows a schematic representation of the enrichment for a sub-population of sense cDNA 3′ fragments representing the 3′ ends of mRNAs using cDNA as the initial template. FIG. 5(A) shows an outline for a method for the enrichment of a sub-population of sense cDNA 3′ fragments using PCR with a tagged (i.e. a first member of an affinity pair, for example, but not limited to biotin) primer complementary to plasmid sequence downstream of the cDNA insert in combination with another primer complementary to the plasmid region upstream of the insert. As a result of this protocol the antisense strands are tagged. Following PCR, the double stranded cDNA products are fragmented and the tagged DNA/DNA hybrids are captured using the second pair of the affinity pair, for example, but not limited to, streptavidin. The sense cDNA 3′ fragments are then eluted from the bound hybrid or the DNA/DNA hybrids are released from the bound vector portion by restriction digest and are denatured into single strands before use. FIG. 5(B) shows an alternate schematic for the enrichment of a sub-population of sense cDNA 3′ fragments. Antisense strands are synthesized from a linearized template using a primer tagged with a first member of an affinity pair (for example, biotin), the primer complementary to the plasmid region downstream of the cDNA insert. The tagged antisense strands are hybridized with small cDNA fragments and treated with a DNA nuclease. Tagged DNA/DNA hybrids are captured using the second member of the affinity pair for example, streptavidin. The sense cDNA 3′ fragments are then eluted from the bound hybrid or the DNA/DNA hybrids are released from the bound vector portion by restriction digest and are denatured into single strands before use. These single stranded cDNA sub-populations direct synthesis from the 3′ end of the coding region towards the 3′ flanking region and thus can be used to isolate 3′ coding and flanking DNA.

FIG. 6 shows a schematic representation for the enrichment of a sub-population of sense cDNA 5′ fragments representing the 5′ ends of mRNAs using cDNA as the initial template. Plasmids containing cDNA inserts are linearized and the antisense strands are synthesized using a primer complementary to the plasmid region downstream of the cDNA inserts. The 3′ ends are then tagged using a first member of an affinity pair, for example biotin, and the tagged cDNAs are hybridized to small fragments of cDNA and treated with a DNA nuclease. Tagged DNA/DNA hybrids are captured using the second member of the affinity pair, for example streptavidin, and the sense cDNA 5′ fragments are then eluted. In this approach the cDNA sub-population corresponds to small single stranded cDNA fragments that direct DNA synthesis from the 5′ end of the coding region and can thus be used to isolate coding sequences and 3′ flanking DNA.

FIG. 7 shows a schematic representation of the enrichment for a sub-population of sense cDNA 3′ fragments representing the 3′ ends of mRNAs, using mRNA as the initial template. The first strands of cDNA are synthesized using an oligo d(T) primer tagged with a first member of an affinity pair, for example, biotin. FIG. 7(A) shows an outline of a method for the enrichment of a sub-population of sense cDNA 3′ fragments. Following second strand synthesis the double stranded cDNAs are fragmented and the tagged 3′ ends are captured with a second member of an affinity pair, for example streptavidin. The sense cDNA 3′ fragments are then eluted. FIG. 7(B) shows an alternate scheme of a method for the enrichment of a sub-population of sense cDNA 3′ fragments. The tagged cDNA is hybridized with small DNA fragments and the DNA/DNA hybrids treated with a DNA nuclease. The tagged 3′ ends are captured with the second member of the affinity pair (e.g. streptavidin). The sense cDNA 3′ fragments are then eluted. These single stranded cDNA sub-populations direct synthesis from the 3′ end of the coding region towards the 3′ flanking region and thus can be used to isolate 3′ coding and flanking DNA.

FIG. 8 shows a schematic representation for the directional cloning of full-length cDNA using plasmid pMUSC 1. pMUSC 1 is isolated as a single stranded circular plasmid to which a single strand opener oligonucleotide (SEQ ID NO: 7) is annealed. The resulting double stranded sequence is digested with Xba I that cleaves the plasmid right after the last dT of the 3′ end of the oligo dT stretch. Polyadenylated mRNA is annealed to the oligo dT stretch of the purified linear plasmid and used as a template to synthesize a first cDNA strand using reverse transcriptase. The first cDNA strand is being synthesized directly on to the 3′ end of the plasmid. The reaction is extended in the presence of MnCl₂ generating a three to five dCs overhang at the 3′ end of the first strand synthesized from capped (full-length) mRNA. A compatible 3′ dGs or 4 dGs overhang can be produced on the plasmid by digesting plasmid-opener oligonucleotide hybrid with Bst API or Bst XI respectively or together. The plasmid is then re-circularized.

FIG. 9 shows an alternate method for the directional cloning of full-length cDNA. An oligo dT primer (for example, SEQ ID NO: 5) with a protruding 5′ sequence complementary to the overhang of a restriction site such as produced by Eco RI, is annealed to mRNA to prime first cDNA strand synthesis. The reaction is extended in the presence of MnCl₂ generating a three to five dCs overhang at the 3′ end of the first strand synthesized from capped (full-length) mRNA. This first cDNA strand can be ligated directly into a plasmid, for example, pHMSL1 or 2 (see FIG. 10) or pMUSC 2 digested with Eco RI and Bst API, which produces a 3′ overhang of three dGs, or Bst XI, which generates a 3′ overhang of four dGs, or into a mixture of plasmid digested with Eco RI and both Bst enzymes.

FIG. 10 shows an outline of an alternate method for the production of 5′ or 3′, sense or antisense, cDNA ends. cDNA is first cloned directionally (for example with Eco RI, or other desired site, being at the 3′ end of the cDNA as described in FIG. 9, however, it is to be understood that the Eco RI site may be placed at the 5′ end of the cDNA and that sense or antisense strands may be used) into pHMSL1(+) and the plasmid is isolated as a single stranded circle. The cDNA sequence is then in the sense orientation with a restriction site, for example the Hind III site, at the 5′ end. A single stranded cDNA end isolated from this vector will correspond to the 5′ end of the mRNA and will be in the sense orientation. The single stranded pHMSL1 or 2 vectors form a double-stranded region by hybridization of the stem-loop region that is located outside of the multiple cloning site and the insert. This region can be digested using various restriction enzymes such as Eco RV, Asp I, Swa I or Bss HII to release the single stranded insert. A restriction site oligonucleotide (“rs oligo”; for example but not limited to SEQ ID NO: 8), complementary to the plasmid region flanking the cDNA insert, which contains a desired flanking restriction'site, for example, Hind III, and an optional triple helix site, is annealed to the single stranded insert. A single stranded DNA nuclease such as S1 nuclease, or a restriction enzyme capable of digesting single stranded DNA may be used to partially digest the single stranded DNA. If the rs oligo contains a triple helix site, a tagged (for example, biotin) triple helix oligonucleotide (Roche Molecular Biochemicals) maybe annealed to the double stranded region consisting of the insert and the rs oligo, and the resulting triple helix complex captured using the second member of an affinity pair, for example streptavidin, and separated from the remaining nucleic acids. If a triple helix is not used, then a tagged rs oligo may be used to capture the annealed DNA. After capture, the double stranded rs oligo is digested with a desired restriction enzyme, for example Hind III, to release the single stranded cDNA ends.

FIG. 11 shows the purification of cDNA ends. cDNA inserts corresponding to tubulin 4 (SEQ ID NO: 16; TUA4), metallothionein (SEQ ID NO: 17; MT2), prohibitin 2 (SEQ ID NO: 18; phb2) and histone 4 (SEQ ID NO: 19; H4) genes are tagged with a first member of an affinity pair, for example, biotin using PCR and then digested with a first restriction enzyme (lanes 1). The biotinylated fragments are captured using the second member of the affinity pair, for example, streptavidin and the non-bound material is eluted (lanes 2). The bound DNA (representing the 5′ ends of the cDNA) is first washed (lanes 3) and then subjected to a second restriction enzyme digest that cleaves the bound DNA at the vector/insert junction releasing the cDNA ends (lanes 4), as outlined in FIGS. 4 and 5.

FIG. 12 shows the enrichment of genomic fragments containing coding regions as demonstrated schematically in FIGS. (1A) and (1B). Circular single-stranded plasmids containing genomic DNA inserts of the Arabidopsis thaliana RAD51 analog, histone 4, interferon-like, prohibitin 2, cytochrome P450 and TATA box bpaf genes are annealed to the denatured 5′ ends of the RAD51 analog. Second strand synthesis and ligation is performed, and single-stranded DNA is removed. Bacteria are transformed and plasmid DNA isolated and restricted with Hinc II. Lane 1 shows the restriction digest for the pHSacX-T1 plasmid without an insert, lanes 2-7 show the restriction pattern for plasmids containing the genomic clones for the prohibitin 2, histone 4, interferon-like, cytochrome P450, TATA box bpaf and RAD51 analog respectively. Lanes 8-10 show the restriction pattern for 3 independent plasmid preparations obtained after enrichment and which display the pattern expected for RAD51 (Lane 7), and lane 11 is the GeneRuler Ladder Mix (MBI Fermentas).

### DESCRIPTION OF PREFERRED EMBODIMENT

The present invention relates to methods of isolating DNA sequences. More specifically, this invention relates to methods of isolating and analyzing coding and flanking regions of genes.

The following description is of a preferred embodiment by way of example only and without limitation to the combination of features necessary for carrying the invention into effect.

Numerous methods have been described in the prior art to isolate and generate gene coding sequences. Although the number of ESTs representing partial gene coding sequences have increased dramatically in public databases, the number of sequences for flanking sequences has not kept pace. The main reason for this discrepancy is that coding sequences can be readily obtained by reverse transcription of mRNA, whereas flanking sequences are buried in non-coding DNA that typically makes up most of the chromosomal DNA.

The present invention provides methods and compositions for the high throughput isolation of DNA fragments, including non-coding regions that flank coding regions of DNA. The methods include enriching for genomic DNA surrounding genes using nucleic acid hybridization, and generating libraries that can be readily screened, sequenced or analyzed. The disclosed methods allow the production of large numbers of gene coding sequences, 5′ or 3′ sequences, both 5′ and 3′ flanking sequences or a combination thereof, as well as methods for the analysis for these sequences on a large scale. The methods disclosed herein provide for the enrichment of DNA containing gene coding and flanking sequences from genomic DNA using nucleic acid hybridization to either DNA or RNA populations, and a means of incorporating gene coding and flanking sequences into convenient cloning plasmids for further manipulation.

The present invention also provides methods for the generation of libraries containing either 5′ regions or 3′ regions of genomic DNA containing coding and flanking sequences. These libraries can serve as templates for high throughput sequencing of promoter sequence tags (PSTs) and 3′ sequence tags (TSTs). Furthermore, since the 5′ or 3′ flanking regions include, or are attached to, a portion of the coding sequence, a plurality of flanking regions, for example promoter regions, can be studied using micro or macro arrays (referred to as “flanking arrays” or “flanking DNA arrays”) using any suitable probe, for example oligonucleotides, cDNA, genomic DNA, RNA, mRNA or protein probes. These probes hybridize to their counterpart coding sequence target within the flanking array or in the case of proteins, to their target DNA binding site. Since the coding sequence targets are attached to a flanking sequence, such an analysis readily identifies flanking regions responsible for expression of the corresponding cDNA or mRNA probes. If proteins are used as probes, then any DNA sequences involved in protein binding may be identified.

Flanking arrays may be used in a variety of ways, for example which are not to be considered limiting in any manner, cDNAs or mRNAs obtained from different tissues, or under a variety of developmental or environmental conditions can be used to probe a flanking array, for example a promoter array, to identify 5′ flanking regions and associated promoters active in the expression of mRNA in the tissues, or under the conditions examined. For example, which is not to be considered limiting in any manner, this high throughput approach can be used to readily identify classes of promoters that are active within specific tissues of an organism, for example, if the organism is a plant, the root tip, root hairs or other portions of the root, the stem, including nodes, internodal tissues and auxiliary buds, meristematic tissues from the root or shoot, leaf, floral tissues or parts thereof including anthers and portions thereof, stigma, style, ovary, calix, petals, fruit and portions thereof, or seed. However, other tissues of a plant or other organism may be used. Furthermore, this high throughput approach may readily identify classes of promoters active within developmental processes within an organism, for example, if the organism is a plant, germination, shoot, root or leave development, flowering, or promoters active in response to environmental factors, including light, dark, temperature, moisture, nutrient supply, wounding, salt, or other environmental stimuli or stresses that may be encountered. Analogous flanking arrays comprising 3′ flanking sequences (3′ flanking array) may also be used to examine similar processes. It is also to be understood that analysis using promoter, or 3′ flanking, arrays may also be performed using flanking regions prepared from any source, including but not limited to animal, insect, bacterial, fungal sources. Therefore, as would be understood by one of skill in the art, this method is not limited to the source of the probes, their labelling, or the DNA sequences used to prepare the flanking array.

Flanking arrays, comprising either 5′ or 3′ flanking sequences may also be used to select for DNA binding proteins that associate with 5′ or 3′ flanking sequences (for example using the method of Bulyk M. L., et al. Nature Biotech, 1999, 17:573-577). In this method, the flanking array is probed with one or more proteins obtained from different tissues, or under a variety of developmental or environmental conditions. This assay identifies 5′ or 3′ flanking regions and associated sequences capable of binding DNA binding proteins. For example, which is not to be considered limiting in any manner, this high throughput approach can be used to readily identify classes of DNA binding proteins that are expressed within specific tissues, or within a range of developmental processes, of a desired organism. Such an analysis may be performed using flanking arrays prepared from any source, including but not limited to plant, animal, insect, bacterial, fungal sources. Therefore, as would be understood by one of skill in the art, this method is not limited to the source of the protein probes, or the DNA sequences used to prepare the flanking array.

Arrays may also comprise a plurality of promoter fragments. A promoter fragment is a 5′ flanking fragment that is not attached to a coding fragment. An array of promoter fragments may be used to select for DNA binding proteins, as described above (e.g. using the method of Bulyk M. L., et al. Nature Biotech, 1999, 17:573-577), or may be used to select for specific regulatory motifs that are present within a promoter region as would be known to one of skill in the art. Similarly, arrays may also comprise a plurality of 3′ untranslated fragments, wherein the 3′ untranslated fragment is not attached to a coding fragment. An array of 3′ untranslated fragments may be used to select for DNA binding proteins that specifically associate with the 3′ untranslated region (for example, using the method of Bulyk M. L., et al. Nature Biotech, 1999, 17:573-577), or may be used to select for specific regulatory motifs that are present within a 3′ untranslated region as would be known to one of skill in the art.

High throughput assays as described herein may also be used to identify promoters that may be repressed or activated within a tissue, or during development, or in response to specific environmental conditions in any test organism. In this embodiment, flanking array are probed using one or more cDNA populations obtained from specific tissues, or during a range of developmental processes. For example, which is not to be considered limiting in any manner, if the test organism is a plant, two or more cDNA populations may be obtained at various stages during flowering, germination, or during light or dark induction, or in response to environmental stresses (for example temperature, salt, wounding. etc.), and these cDNA populations may be compared using a flanking array obtained from the same or related organism. Analogous comparisons may be made using methylated and non-methylated libraries in order to determine the methylation pattern of genomic DNA. Any difference(s) in the expression patterns detected within the flanking array may be used to identify for example, promoters that are active or repressed within the selected tissue, or under the tested condition. As would be evident to one of skill in the art, any differences in gene expression can be detected using this method. Furthermore, DNA from any test organism may be used to prepare the flanking array or cDNA or mRNA probes. Therefore, the present invention is not limited by the source of the DNA or probe populations.

The methods of present invention may be used to generate 5′ and 3′ flanking sequences on a large scale. This permits high throughput analysis of flanking sequences, for example high throughput sequencing of any flanking sequence tag (FST), including PSTs, and TSTs. In addition to the analysis of flanking sequences using flanking arrays discussed above, it will be evident to someone skilled in the art that the availability of large numbers of flanking sequences may also be characterized using known methods, for example which are not to be construed as limiting in any manner, high throughput sequencing, high throughput analysis of 5′ flanking sequences using the one-hybrid system (Joachim J. L. and Herskairtz I. Science, 1993, 262:1870- 1874), or Southwestern analysis (Philippe, J., Meth. Mol. Biol., 1994, 31:349-361) to identify promoter binding proteins. The availability of large numbers of flanking sequences permits high throughput analysis using large scale transformation of organisms, for example but not limited to, model organisms Arabidopsis or zebra fish, using transient assays, for example virus induced gene silencing (VIGS; Baulcombe, D. C., Curr. Opin. Plant Biol., 1999,2:109-113), biolistics, or agroinfiltration (Yang, Y., et al., Plant J., 2000,22:543-551).

It is also contemplated that using the methods described herein, genomic DNA fragments from any source that comprise a gene coding sequence may also be used to populate arrays (genomic arrays). In this embodiment, such a genomic array may be probed by a population of cDNAs to identify, on a high throughput scale, genomic counterparts of the probe cDNA population. The genomic counterparts may be from the same, or from a different source as the population of cDNA probes. As will be evident from the methods described below, the identified genomic counterparts are available within a form that is readily manipulated or sequenced and suitable for high throughput processing, for example sequencing.

Therefore, the proposed invention is directed to methods to select for gene coding and flanking DNA regions from genomic DNA and further provides methods to analyze, characterize, and sequence these sequences. Although the methods below exemplify approaches for selecting genomic coding and flanking regions that are suitable for high throughput processing, it will be apparent to a person skilled in the art that these methods are also applicable on a scale that can be reduced to a single DNA fragment or RNA for which flanking sequences are desired. Similarly, the methods described herein exemplify the isolation of gene coding and flanking sequences from genomic DNA, but it will be obvious to persons skilled in the art that many of these approaches are also applicable to cDNA.

By “genomic DNA” it is meant DNA found in living organisms such as chromosomal or extra-chromosomal DNA, typically genomic DNA comprises coding and noncoding regions, 5′ regulatory regions, scaffold attachments regions, untranslated regulatory sequences, enhancers, silencers, protein binding sequences, mRNA stability determinants or processing signals, localization signals, introns, exons, polyadenylation sequences, transposons, retrotransposons, repeats, microsatellites, 3′ untranslated regulatory regions, however, the presence of these regions within a genomic DNA fragment will depend on the size of the DNA fragment. Genomic DNA fragments are pieces of genomic DNA that maybe generated enzymatically, chemically or physically, and comprise a portion of, or one or more of the regions, as defined above.

By “genomic library” it is meant a collection of genomic DNA fragments which are cloned in a suitable vector such as a plasmid, bacteriophage vector, or viral vector capable of transfecting eukaryotic cells.

By “gene coding region” it is meant the sequence of a gene which is transcribed into RNA. The gene coding region can include introns and the 5′ and 3′ untranslated regions. These untranslated regions may contain regulatory signals capable of effecting RNA processing, RNA stability, or gene expression as known to those of skill in the art. A gene coding fragment is a portion of a gene coding region. A gene coding sequence refers to the nucleotide sequence of the gene coding fragment or region.

By “gene flanking region” it is meant sequences that reside upstream or downstream of a gene coding sequence and that are not typically transcribed into RNA. A gene flanking fragment is a portion of a gene flanking region. A gene flanking sequence refers to the nucleotide sequence of the gene flanking fragment or region.

By “5′ flanking region” it is meant a DNA sequence that is 5′ of a gene coding region. A 5′ flanking region may include regulatory signals capable of effecting gene expression, for example but not limited to regulatory sequences, scaffold attachments regions, enhancers, silencers, protein binding sequences, a promoter. A 5′ flanking fragment is a portion of a 5′ flanking region. A 5′ flanking sequence refers to the nucleotide sequence of the 5′ flanking fragment or region.

By “3′ flanking region” it is meant a DNA sequence that is 3′ of a gene coding region. A 3′ flanking region may contain regulatory signals capable of effecting gene expression. A 3′ flanking fragment is a portion of a 3′ flanking region. A 3′ flanking sequence refers to the nucleotide sequence of the 3′ flanking fragment or region.

By “regulatory sequence”, “regulatory signal” or “regulatory region” it is meant DNA sequence which is involved in modulating gene expression. Typically, regulatory sequences are found in relatively close proximity to genes and are part of the genomic upstream, downstream, or upstream and downstream gene sequence, however, regulatory sequences may also be localized in introns. Regulatory regions may include silencing or enhancing elements, or sequences that effect RNA stability, or that direct tissue specific, developmental, or environmental gene expression. Regulatory sequences may also include promoter regions, for example constitutive or inducible promoters, core promoter sequences, protein binding sequences, and other regulatory elements as would be recognized by one of skill in the art. As defined herein, a regulatory sequence, regulatory signal, or regulatory region are typically, but not always, within a flanking sequence.

By “promoter region” it is meant a nucleotide sequence which is involved in directing gene expression. A promoter region is typically found in the genomic 5′ flanking region of a gene, and is typically within or adjacent to a 5′ flanking fragment.

By “promoter fragment” it is meant a fragment of the promoter region. A promoter fragment is a 5′ flanking fragment that is not attached to a coding fragment.

An array refers to a collection of nucleic acids, of more than 5, preferably more than 10, or more preferably more than 50 nucleic acids that are immobilized or crosslinked to a substrate or matrix, for example but not limited a glass plate, microplate, filter paper, charged membrane or nylon. An array comprises at least 10 nucleic acid fragments. An array includes either a microarray and a macroarray. An array comprising a population of genomic DNA fragments is referred to as a “genomic array”. A “flanking array” (or “flanking DNA array”), refers to an array comprising a population of either 5′ flanking fragments, 3′ flanking fragments, or a combination thereof. These flanking fragments may or may not have coding sequence attached.

A cDNA represents a copy of a gene coding sequence, therefore cDNAs are typically used to screen for a corresponding gene coding, and adjacent genomic, sequences (for example regulatory regions) by nucleic acid hybridization between genomic DNA and the cDNA of interest. Typically, a library consisting of genomic DNA fragments is cloned in an appropriate cloning vector, and then screened with a labelled nucleic acid probe corresponding to a unique cDNA. In this manner genomic DNA libraries may be screened with populations of cDNA, however, it is very difficult to sort out which genomic DNA corresponds to which cDNA probe. Furthermore, establishing the orientation, location and composition of the coding and regulatory sequences within individual genomic clones would require an enormous effort if applied on a large scale. The present invention provides methods for overcoming these deficiencies in these prior methods.

By “cDNA” it is meant DNA complementary to mRNA. This cDNA is typically synthesized from mRNA using the enzyme reverse transcriptase. Fragments of cDNA represent pieces of cDNA generated by partial reverse transcription, enzymatic digestion, chemically or physically, for example. The 5′ end of a cDNA corresponds to the 5′ end of the template mRNA and the 3′ end of a cDNA corresponds to the 3′ end of the template mRNA. A full-length cDNA is a cDNA that contains the entire gene coding sequence. A partial cDNA is a cDNA that contains only part of the gene coding sequence.

By “cDNA library” it is meant a collection of cDNAs which is cloned into a vector such as a plasmid or bacteriophage vector. A cDNA library reflects the sequence and composition of the template mRNAs from which it was reverse transcribed. A full-length cDNA library is a library that is enriched in full-length cDNAs.

By “cDNA population” it is meant a collection of cDNAs. The cDNA population can be a reflection of the template mRNA population or can result from enrichment, normalization or subtraction of a cDNA library relative to one or more other cDNAs.

By “enrichment” it is meant a process which increases the proportion of a particular nucleic acid or group of nucleic acids.

By “cDNA sub-population” it is meant an enriched collection of cDNAs or cDNA fragments.

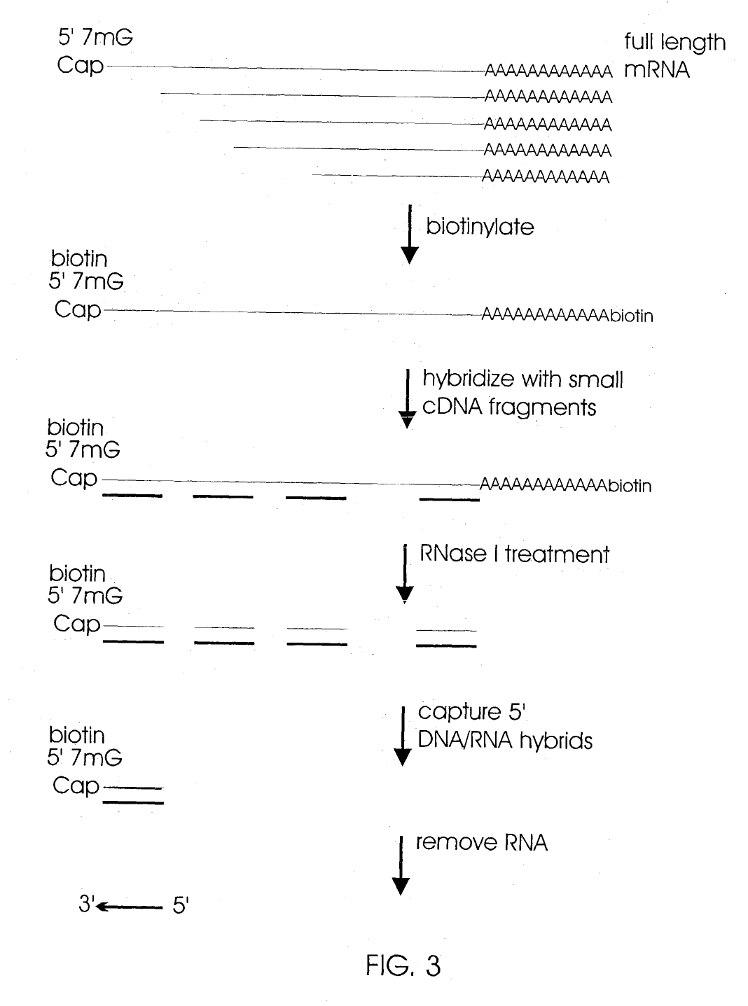

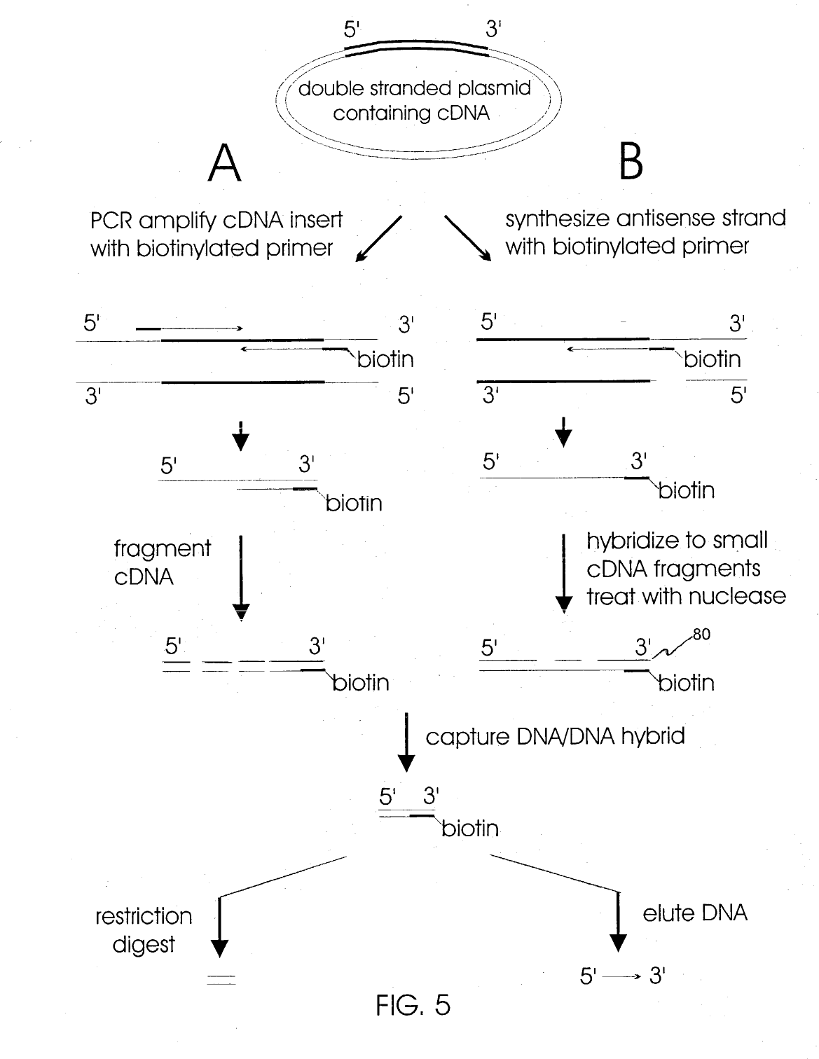

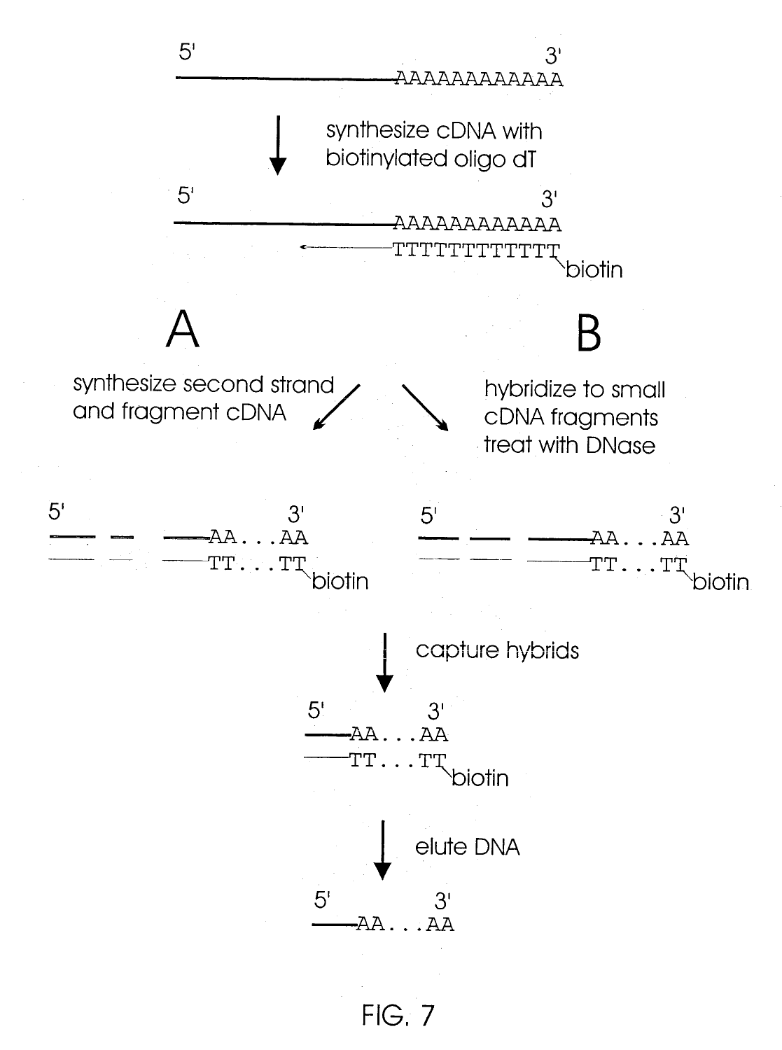

By “cDNA 5′ ends” it is meant a collection of cDNA fragments that are enriched for fragments that correspond to the 5′ end of mRNA sequences. Preferably, the mRNA is full-length (see below, and FIGS. 3, 4, and 6). By “cDNA 3′ ends” it is meant a collection of cDNA fragments enriched for fragments corresponding to the 3′ end of mRNA sequences. Preferably, the mRNA is full-length (see below and FIGS. 5 and 7). These cDNA 5′, or 3′, ends may be used to generate single stranded cDNAs in the same (sense), or an opposite (antisense), orientation as the mRNA. Preferably, the cDNA 5′ and 3′ ends are single stranded cDNAs.

By “RNA end” it is meant a collection of mRNA fragments that are enriched for either a 5′ or a 3′ end of mRNA sequences. Preferably, the initial mRNA is full-length.

By “sub-population of sense cDNA 5′ ends” it is meant a collection of cDNAs, preferably enriched for single stranded cDNA fragments, that correspond to the 5′ end of mRNA, which are in the same orientation as the mRNA. This sub-population of sense cDNA 5′ ends can be used in the methods of the present invention to isolate coding and downstream sequences.

By “sub-population of antisense cDNA 5′ ends” it is meant a collection of cDNAs, preferably enriched for single stranded cDNA fragments, corresponding to the 5′ end of mRNA, which are in the opposite orientation of the mRNA. This sub-population of antisense cDNA 5′ ends can be used in the methods of the present invention to isolate coding and upstream sequences.

By “sub-population of sense cDNA 3′ ends” it is meant a collection of cDNAs, enriched for single stranded cDNA fragments, that correspond to the 3′ end of mRNA, which are in the same orientation as the mRNA. This sub-population of sense cDNA 3′ ends can be used in the methods of the present invention to isolate coding and downstream sequences.

By “sub-population of antisense cDNA 3′ ends” it is meant a collection of cDNAs, enriched for single stranded cDNA fragments, corresponding to the 3′ end of mRNA, which are in the opposite orientation of the mRNA. This sub-population of sense cDNA 3′ ends can be used in the methods of the present invention to isolate coding and upstream sequences.

By “flanking sequence tag” or “FST”, it is meant a partial DNA sequence of a gene flanking region, therefore an FST may comprise regulatory signals capable of effecting gene expression, for example but not limited to one or more regulatory sequences, enhancers, silencers, protein binding sequences, a promoter, a scaffold attachments region, or portions thereof. An FST may comprise a promoter sequence tag (PST), a 3′ sequence tag (TST), or a portion thereof.

By “promoter sequence tag” or “PST”, it is meant a partial DNA sequence of a sequence upstream of a gene coding region. A PST may include sequences which control the expression of a coding region by providing recognition sequences for RNA polymerase and/or other factors required for transcription to start at a particular site. An FST may be a PST.

By “3′ sequence tag” or “TST”, it is meant a partial DNA sequence of a sequence downstream of a gene coding region. A TST may comprise regulatory elements that modulate or terminate gene expression as would be recognized by one of skill in the art. An FST may be a TST.

By “affinity pair” it is meant a group of two or more compounds that have the ability to bind with other members of the group in a reversible or non-reversible manner. An affinity pair may comprise two or more molecules that can bind with each other, for example which is not to be construed as limiting in any manner, biotin and streptavidin, complementary stands of a nucleic acid, or a triple helix. It is to be understood that an affinity pair may comprise more than two different members. One member of an affinity pair may be attached to a nucleic acid for which separation is desired from a population of other compounds (a tagged nucleic acid), and the second member of the affinity pair may be attached to a substrate, for example but not limited to, a magnetized bead, chromatographic matrix, filter paper, or a surface of a well, for example the well of a microplate. In this manner, the tagged nucleic acid, after associating with the second member of the affinity pair, may be selectively removed from a heterogeneous mixture of compounds by using an appropriate separation strategy, for example centrifugation, column chromatography or washing. The tagged nucleic acid may then be recovered from the second member of the affinity pair by disrupting the association between the affinity pair using standard methods as would be known to one of skill in the art.

By “vector” it is meant a nucleic acid sequence which may be used to transform or transfect an organism of interest. A vector typically comprises one or more cloning sites so that a nucleotide sequence of interest may be introduced into the vector prior to transformation or transfection. Examples of vectors, which are not to be considered limiting in any manner, include plasmids, or linear phages.

Methods for Obtaining Gene Coding, 5′, or 3° Flanking Fragments

Methods are provided herein that are directed to producing populations of genomic DNA that are enriched in either gene coding fragments, or gene coding fragments and 5′ or 3′ flanking fragments, or a combination thereof. These enriched populations may be characterized directly and sequenced to elucidate information regarding these regions. Gene coding fragments comprising 5′ and 3′ flanking fragments may also be used as probes to obtain longer regions of upstream and downstream regions of desired genes for further analysis of regulatory regions and associated elements. Furthermore, enriched populations of gene coding fragments comprising 5′ and 3′ flanking fragments may be used for gene expression analysis in array (both microarray or macroarray) analysis. Similarly, gene coding fragments may be used in array analysis to readily identify and manipulate genomic counterparts of cDNA populations. In addition, the availability of flanking arrays, comprising either 5′ flanking fragments, or 3′ flanking fragments, or a combination thereof, allows the high throughput analysis of regulatory activities of these regions, or, if desired, associated binding proteins.

Although double stranded plasmids or denatured double stranded plasmids can be used as templates for hybridization in the present invention, it is preferred that single stranded plasmids containing genomic DNA be used for the hybridizations described below. As evident to one of skill in the art, other approaches can be used to achieve enrichment. An example of an alternative approach, which is not to be construed as limiting in any manner, comprises the use of RecA-mediated affinity capture which allows enrichment of genomic DNA using double stranded plasmids as templates for hybridization (Zhumabayeva, B., et al., BioTechniques, 1999, 27:834-845).

Although not described implicitly in the present invention, one of skill in the art will appreciate that since cDNA reflects the mRNA expression occurring in a cell, then a plurality of cell types functioning under varied living conditions can be used to generate the mRNA (or derived cDNA) templates used for hybridization as described herein.

General Overview of Method

Methods, directed to enriching genomic DNA fragments containing either gene coding fragments, or gene coding fragments associated with 5′ or 3′ flanking fragments using cDNA or mRNA are presented below.

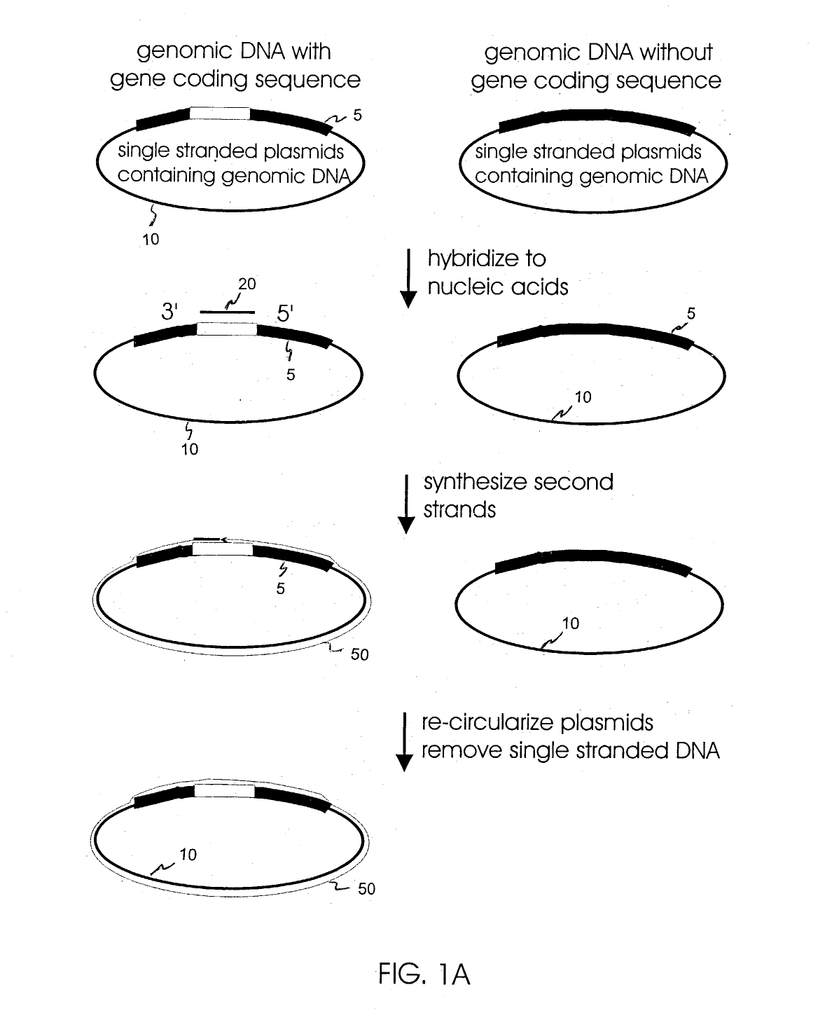

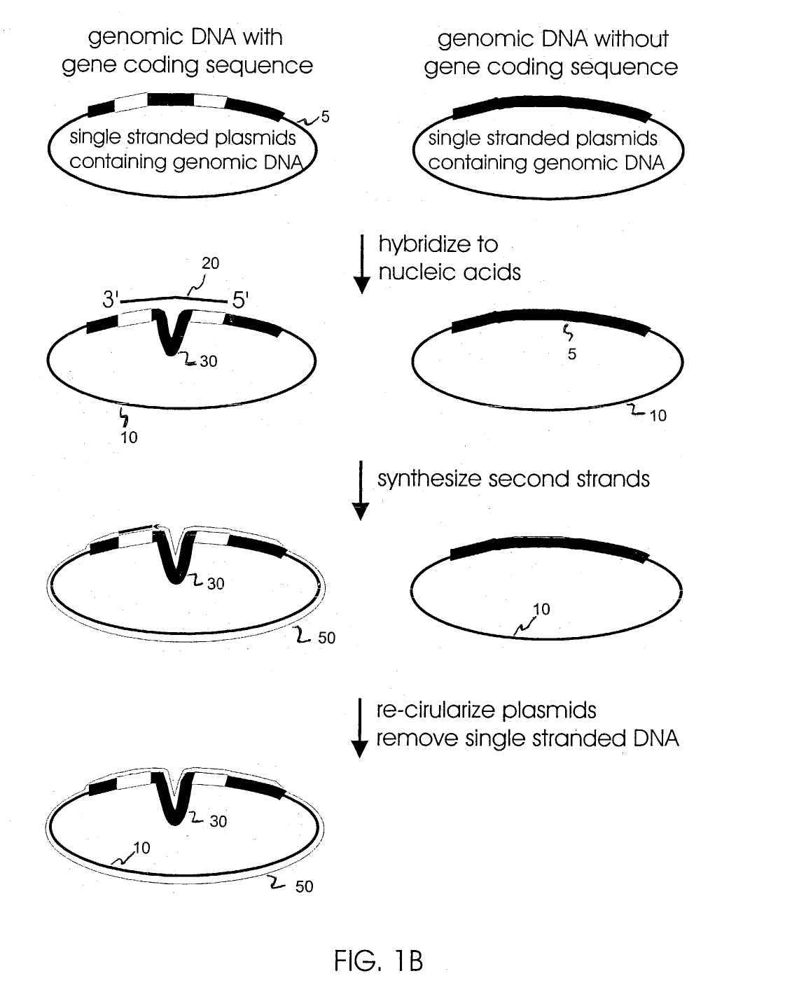

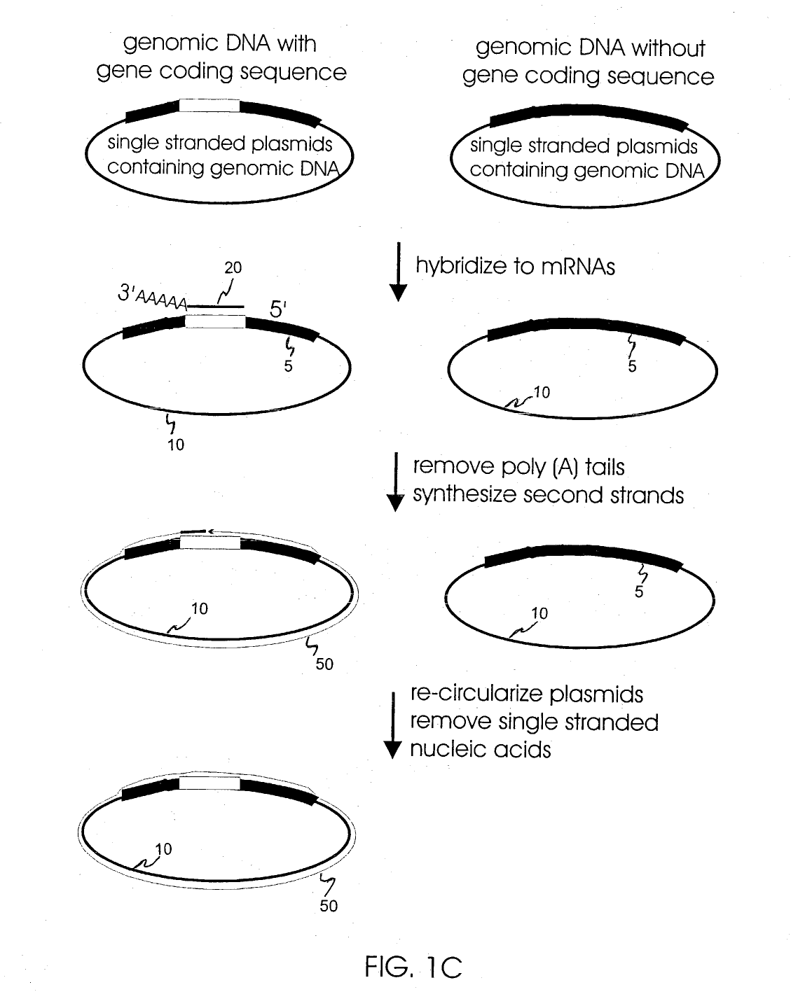

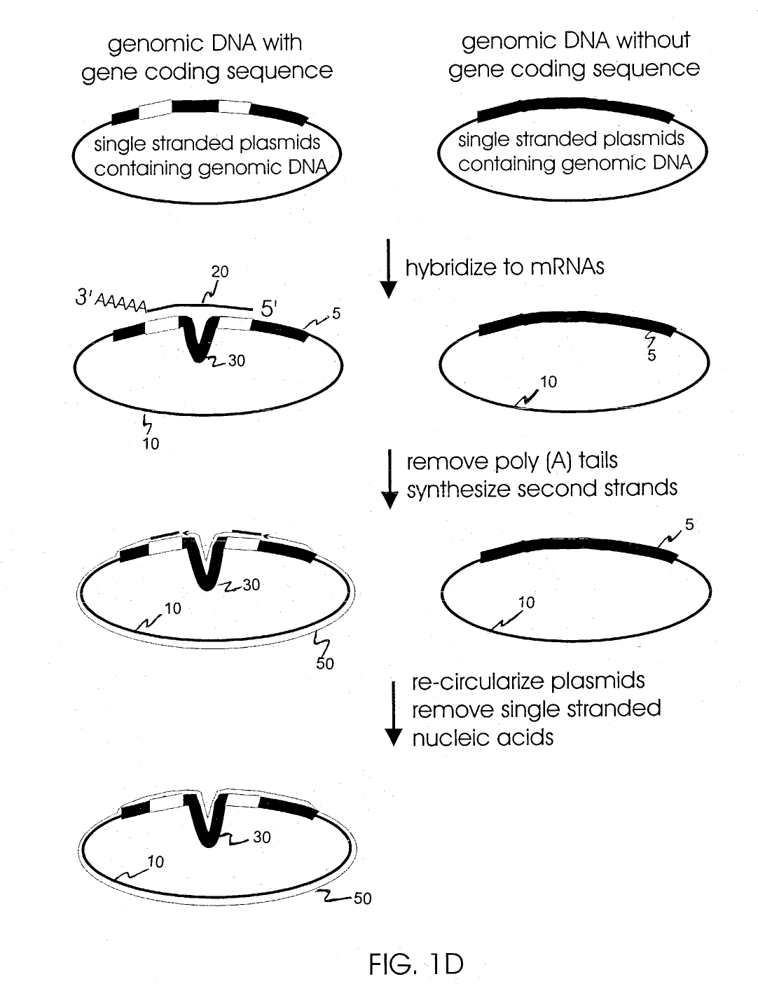

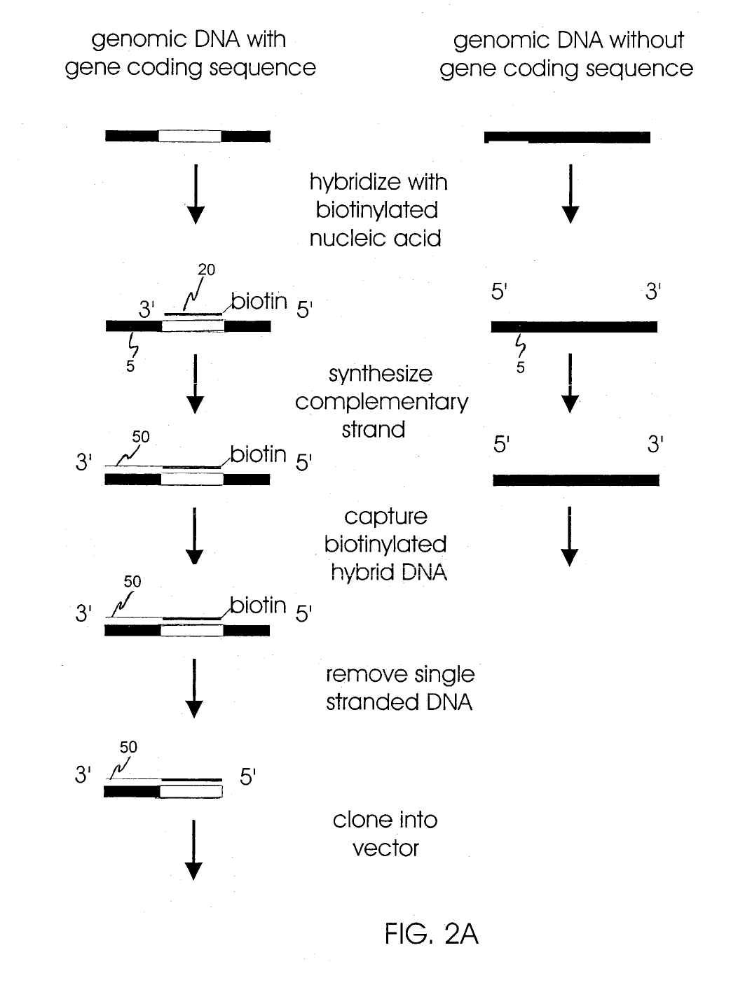

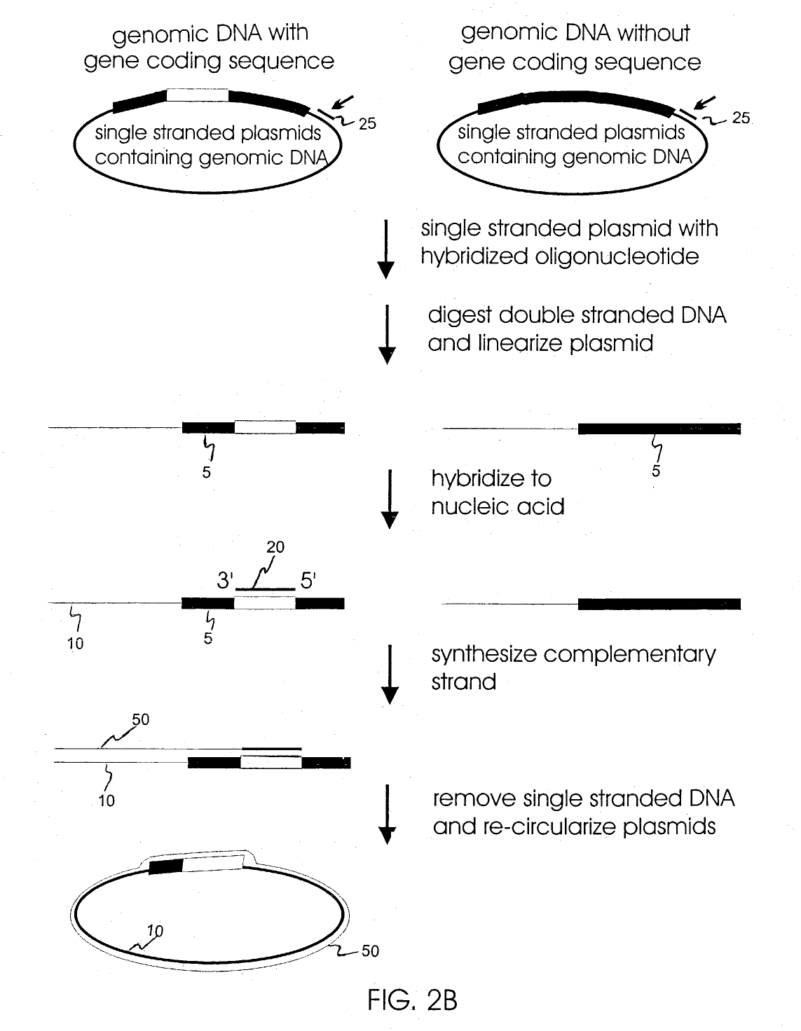

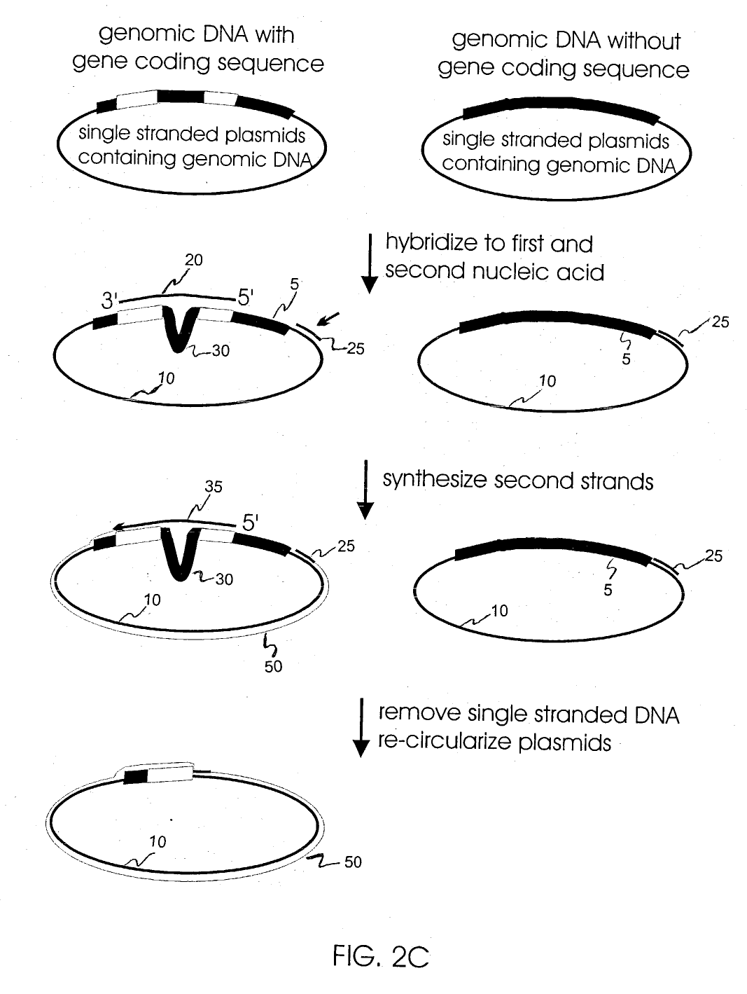

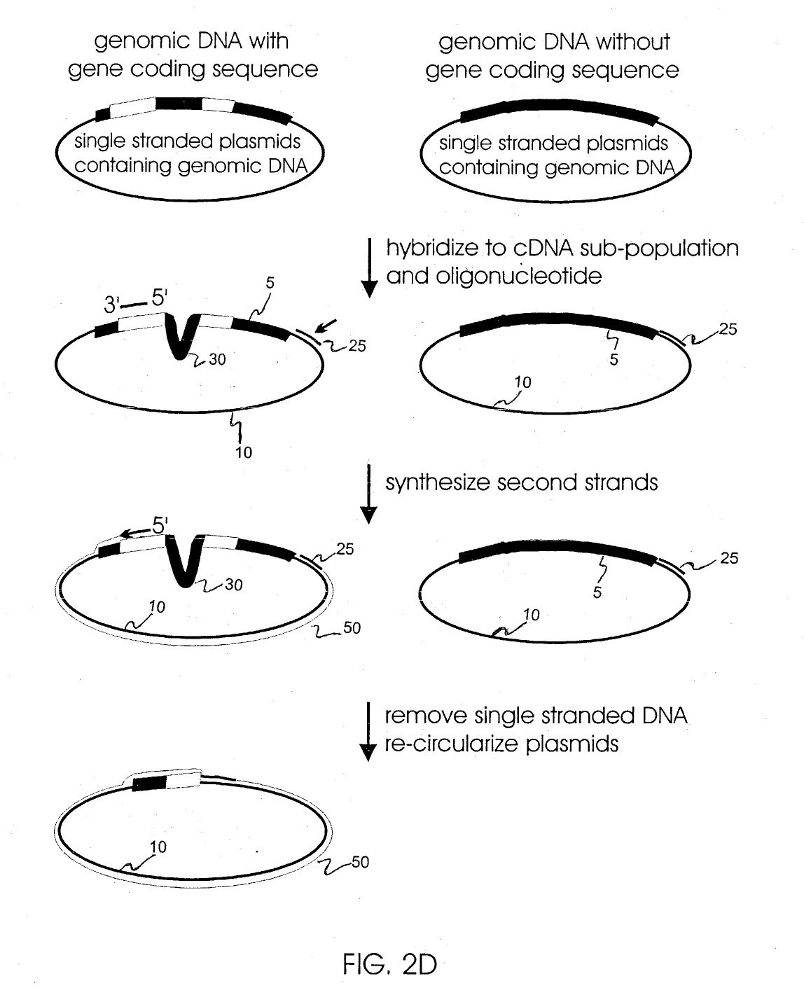

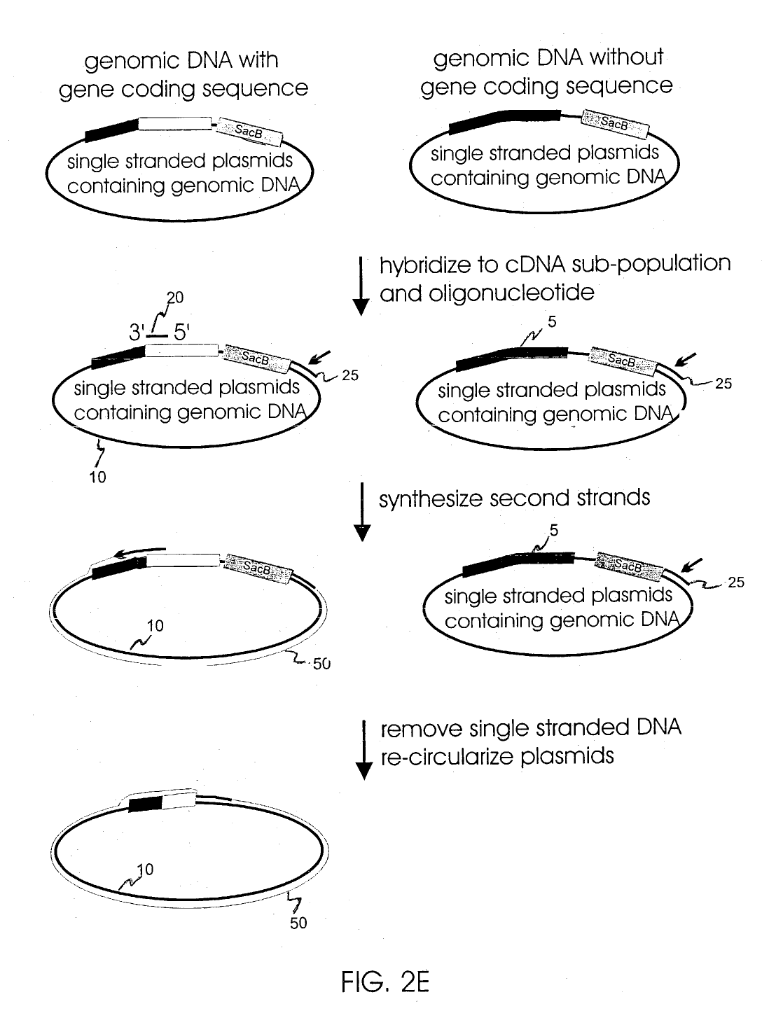

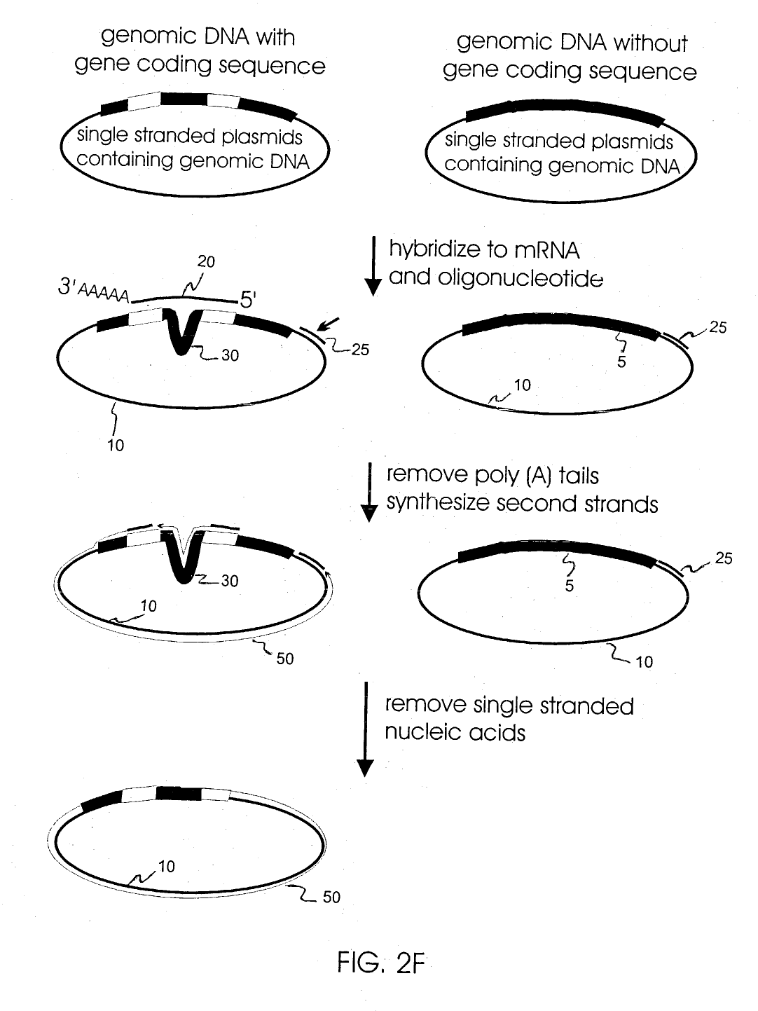

With reference to FIG. 1(A), there is provided a method comprising preparing a plurality of genomic DNA fragments (5; template DNA) and introducing these fragments into cloning vectors, for example but not limited a plasmid or phage, which can produce single stranded DNA, and producing a population of single stranded DNA (10). However, it is to be understood that double stranded DNA or denatured double stranded DNA can be used as templates for hybridization in the present invention. Single stranded DNA containing genomic DNA fragments (5) are then hybridized to one or more nucleic acids (20), for example but not limited to cDNAs (FIGS. 1(A), (B)), 5′ or 3′ cDNA ends, genomic DNA, synthetic DNA, or mRNAs (FIGS. 1(C) and (D)), and a second strand (50) synthesized from the single stranded DNA using the hybridized nucleic acid (20) as a primer for DNA synthesis. Any remaining single stranded nucleic acid is removed, for example, but not limited to, by using an appropriate nuclease for example a DNA nuclease, RNA nuclease or a combination thereof, and the remaining vectors reconstituted. This step removes any vectors comprising genomic DNA fragments that lack any gene coding region (see right hand side of FIG. 1(A)). The final population of vectors comprises both fragments of genomic DNA containing gene coding fragments, and gene coding fragments associated with 5′ or 3′ flanking sequences suitable for further manipulation. Variations on this method are provided in FIGS. 1(B) to 1(D), and 2(A) to 2(F) as described below.

The above method, described with reference to FIGS. 1(A) to 1(D), and FIGS. 2(A) to 2(C) and variations thereof, illustrate how gene coding, and flanking fragments can be obtained using a nucleic acid (20) and a vector containing genomic DNA (10). These methods may be modified to further enrich for the production of 5′ and 3′ flanking sequences by using a population of cDNA fragments enriched for 5′ or 3′ ends, for example FIGS. 2(D) and 2(E) that correspond to the 5′ or 3′ ends of mRNAs respectively, and mRNA (FIG. 2(F)).

With reference to FIG. 2(A), this method comprises preparing a plurality of single stranded or double stranded genomic DNA fragments (5). These genomic DNA fragments are hybridized to one or more nucleic acids (20), for example cDNA, 5′ or 3° cDNA ends, genomic DNA, synthetic DNA, or mRNA, which are preferably tagged with one member of an affinity pair, and a second strand (50) is synthesized from the single stranded genomic DNA template using the hybridized nucleic acid as a primer for DNA synthesis. Tagged DNA/DNA fragments are captured using the second member of the affinity pair. Any remaining single stranded nucleic acid is removed, for example, but not limited to, by using an appropriate DNA nuclease, RNA nuclease or both a DNA and an RNA nuclease. The final population of DNA comprises both genomic DNA containing gene coding fragments, and gene coding sequences associated with 5′ or 3′ flanking fragments suitable for further manipulation.

The method as described above can be further modified by enriching specifically for 5′ or 3′ gene flanking fragments. With reference to FIGS. 2(B)-(F), the present invention provides a method for enriching for 5′ or 3′ gene flanking fragments comprising, preparing a plurality of genomic DNA fragments (5), introducing these fragments into cloning vectors which can produce single strands of DNA (however, double stranded DNA or denatured double stranded DNA may also be used), and producing a population of single stranded DNA (10). The single stranded DNA (10) are then hybridized to two nucleic acids, which are preferably, but not necessarily, single stranded:

a first nucleic acid (25) comprising an unmodified or modified DNA oligonucleotide (see FIGS. 2(B)-(F)) which is complementary (the “COP oligo”) to the vector, adjacent to one end of the genomic insert. Preferably, the first nucleic acid is chemically modified at its 3′end; and

a second nucleic acid primer (20) comprising, but not limited to, a single stranded cDNA, genomic DNA, synthetic DNA (e.g. FIGS. 2(B)-(C)), 5′ or 3′ cDNA ends (see FIGS. 2(D) and 2(E)), or mRNA (see FIG. 2(F)).

If a modified DNA oligonucleotide (25) is used, then a second strand of DNA (50) may be synthesized from the vector using the hybridized nucleic acid (20) as a primer for the DNA synthesis (see FIGS. 2(C)-(F)). If an unmodified DNA oligonucleotide (25) is used, then the double stranded region comprising the unmodified oligonucleotide may be cleaved with a restriction enzyme (see FIG. 2(B)). Following second strand synthesis, any single stranded nucleic acid (e.g. 30, 35, FIGS. 2(C) and (F)) is removed, for example, but not limited to, with one or more nucleic acid nucleases, for example a DNA nuclease, an RNA nuclease, or a combination thereof, and the vector reconstituted. Alternatively, following second strand synthesis the plasmids can be linearized using a restriction site in the “COP” oligonucleotide (eg. Sfi I-A in COP-2, SEQ ID NO: 28) followed by treatment with a DNA polymerase which can blunt end DNA for example, but not limited to, T4 DNA polymerase, T7 DNA polymerase or the Klenow fragment of DNA polymerase I. To further enrich for plasmids where the intervening single-stranded DNA has been removed, the above steps (see FIGS. 2(C) to 2(F)) can be performed using a plasmid containing the sacB gene for levansucrase from Bacillus subtilis (Steinmetz, M., et al., Mol. Gen. Genet., 1985,200:220-228). In vector pHSacX-T1 (see below) the sacB gene has been inserted in the plasmid region located between the binding site of the “COP” oligonucleotide and the cloned genomic DNA insert (see FIG. 2(E)) such that the sacB gene remains single-stranded and can be removed as described above. In situations where the sacB gene is not removed along with the other single-stranded DNA, bacteria containing plasmids with the sacB gene can be selected against by growing them on sucrose (Quandt, J. and Hynes, M. F., Gene, 1993,127:15-21). The final population of vectors comprise gene coding fragments, and gene coding fragments associated with 5′ or 3′ flanking sequences suitable for further manipulation. However, by removing 5′ portions of the second strand of DNA (20) and portions of the single stranded vector that remain single stranded during this procedure, the population of vectors are enriched in genomic DNA comprising flanking sequences.

Preparation of Template DNA (5)

The genomic DNA fragments (5) used in the method of the present invention can be generated by a partial or complete restriction enzyme digest as would be known to one of skill in the art such as, but not limited to a partial Sau 3A digest, physical means, PCR, or other methods of fragmentation known in the art. The genomic fragments may be of any suitable length. The genomic fragments are preferably introduced into a cloning vector such as a plasmid (for example, but not limited to, pH-T1, pHSX-T1, pHSacX-T1, see below) or phage, by DNA ligation, recombination or other methods of joining DNA known in the art. Single stranded DNA can be generated using any suitable method, for example but not limited to Gene II/Exonuclease III treatment, or by using vectors known in the art (for example but not limited to pHelix plasmid; Roche Molecular Biochemicals or derivatives) which allow such synthesis. Preferably, in organisms with genomes containing large amounts of repetitive DNA, the genomic library would be “pre-filtered” by using methods known in the art which differentiate repetitive genomic DNA from non-repetitive genomic DNA. Such methods include, but are not limited to methods employing methylation sensitive enzymes (eg. Pst I or Mbo I) and/or McrA/BC E. coli strains which can discriminate against methylated genomic DNA, thus discriminating against repetitive DNA and enriching for actively transcribed genomic DNA (Rabinowicz, P. D. et al., Nature Genet., 1999, 23:305-308; Craig, J. M. et al., Hum. Genet. 1997, 100:472-476). Other examples of approaches which can be used to enrich for specific parts of the genome which are not to be construed as limiting in any manner, involve the production of PCR-derived sub-populations of genomic DNA (Lisitsyn, N. and Wigler, M., Science, 1993, 259:946-951), or using enzymes which can discriminate among the different transcriptional states of the genome (Spiker, S., et al., Proc. Natl. Acad. Sci. USA, 1983, 80:815-819). Furthermore, genomic DNA can be prepared following the methods as outlined, for example, in FIGS. 1(A) to (D). The final reconstituted vectors, for example but not limited to a circularized population of plasmids, may also be used as a template and hybridized to 5′ or 3′ cDNA ends to enrich for flanking fragments. These and other related methods could be used in conjunction with the present invention.

Preparation of the Nucleic Acid Primer (20; the “Second Nucleic Acid”)

The second nucleic acid primer (20) typically comprises one or more cDNAs, genomic DNAs, synthetic DNAs, 5′ cDNA ends, 3′ cDNA ends, mRNA, RNA, or a combination thereof. Preferably, the second nucleic acid primer corresponds to a coding region of a genomic DNA. The use of short second nucleic acid primers within some of the methods as described herein will aid in the enrichment of 5′ or 3′ flanking fragments.

The second nucleic acid (20) can be full, partial or fragmented and generated by restriction enzyme digest, physical means, PCR, or other methods of fragmentation known in the art, and selected from cDNA, 5′ or 3′ cDNA ends (see below, and FIGS. 3-7), genomic DNA, synthetic DNA, RNA or mRNA. The second nucleic acid can be double stranded and denatured prior to hybridization, or single stranded, for example mRNA or cDNA generated by reverse transcription from rnRNA using methods well known in the art, or by single strand DNA synthesis using techniques (eg. Gene II/Exonuclease III treatment) or vectors known in the art (eg. pHelix plasmid; Roche Molecular Biochemicals) which allow such synthesis. If it is desired to enrich the genomic fragments for 5′ and 3′ flanking fragments, then 5′ or 3′ cDNA or mRNA ends may be used as the nucleic acid primer (20). The preparation of 5′ and 3′ cDNA ends is described in more detail below with reference to FIGS. 3 to 7. Since genomic DNA can be cloned in a vector in either orientation, with double stranded cDNA, it is anticipated that both cDNA strands could potentially hybridize. Conditions for DNA/DNA hybridizations are well known in the art (for example Ausubel, F. M., et al., eds. Current Protocols in Molecular Biology, 2 vols., 1984, 1994, Wiley & Sons Inc.).

If the second nucleic acid (20) is mRNA, the mRNA can be isolated by methods well known in the art. For the purpose of specifically isolating gene coding and flanking sequences, the mRNA would preferably consist of full-length mRNA isolated using known methods (for example, Carninci, P. and Hayashizaki, Y., Methods Enzymol., 1999, 303:19-44). By obtaining full-length mRNA, ends that correspond to the 5′ region of the mRNA can be produced. The use of mRNA as a second primer would reduce ribosomal RNA and decrease the possibility of introns occurring in the final plasmids containing the genomic DNA. The use of full-length mRNA is not required however, especially if the method is employed to isolate genomic sequences flanking ribosomal genes for example. Conditions for RNA/DNA hybridizations are well known in the art.

The nucleic acid used as a primer (the second DNA; 20) to generate flanking sequences as outlined in FIGS. 2(A) to (E), can be synthesized directly from mRNA using a reverse transcriptase as is well known in the art. To obtain 5′ or 3′ flanking sequences, the appropriate cDNA strand (antisense or sense respectively) can also be synthesized from a plasmid preferably containing a directional full-length cDNA library, produced for example but not limited to, asymmetric PCR, (Rudi, K., et al., BioTechniques, 1999, 27:1170-1177), using a primer complementary to the plasmid sequence flanking the insert and Taq polymerase. The cDNA primer can also be obtained by DNA synthesis from single stranded plasmids containing a directional cDNA library (for example, Ausubel, F. M., et al., eds. Current Protocols in Molecular Biology, 2 vols., 1984, 1994, Wiley & Sons Inc.) or by other methods known to one skilled in the art.

The COP Oligo (25)

The DNA oligonucleotide complementary to the plasmid adjacent to the insert (the first nucleic acid, 25; see FIGS. 2(B)-(F)) will vary in sequence based on the plasmid being utilized and the restriction enzyme chosen. The oligonucleotide COP-1 identified in SEQ ID NO: 1 serves as a non-limiting example for use with the pHelix plasmid, whereas the oligonucleotide COP-2 identified in SEQ ID NO: 28 serves as a non-limiting example for use with the plasmids pHSX-T1 and PHSacX-T1. In SEQ ID NO: 1 and SEQ ID NO: 28, the COP oligonucleotides are designed to contain a restriction endonuclease site. This site can be used to linearize a single stranded vector prior to hybridization with the second nucleic acid primer (20) by annealing the COP oligonucleotide to a single stranded vector followed by restriction digest (FIG. 2(B)), or can be used later to facilitate reconstitution of the vector, for example linearizing the plasmid following second strand synthesis and treatment with a DNA polymerase to remove single-stranded DNA and allow plasmid re-circularization, or both.

To prevent extension by the DNA polymerase from the COP oligonucleotides (25), the 3′ end of the oligonucleotide is preferably modified (eg. addition of amine-, thiol- or other group) using methods known to one skilled in the art. If the COP oligonucleotide is not chemically modified, the linearized vector containing the genomic DNA may be separated from the COP oligonucleotide (25) prior to hybridization using denaturing gel electrophoresis or column chromatography for example, as known to a person skilled in the art. Since genomic DNA can be cloned in the vector in either orientation, it is anticipated that the use of a given COP oligonucleotide will cause linearization or block second strand synthesis at either the 5′ or the 3′ end of the genomic DNA insert approximately on an equal basis. When the end of the genomic DNA insert next to the COP oligonucleotide binding site is the 3′ end, second strand synthesis will occur up to the oligonucleotide producing a double and single stranded vector. The single stranded regions will be eliminated by nuclease (DNA, RNA or both) treatment. Conditions for DNA/DNA hybridization using oligonucleotides are well known in the art (for example, Ausubel, F. M., et al., eds. Current Protocols in Molecular Biology, 2 vols., 1984, 1994, Wiley & Sons Inc.).

Second Strand Synthesis: in Absence of COP Oligo

Synthesis of the second strand (50) of the vector, using the hybridized second nucleic acid primer (20), can be done with any suitable polymerase as would be known in the art. Preferably the polymerase is a DNA polymerase. If the second nucleic acid primer is RNA, then it is preferred that a RNase, for example but not limited to RNase H, be included along with the polymerase.

To allow DNA synthesis from the mRNA primer, the poly (A) tail of the mRNA is first removed using a RNA nuclease (eg. RNase I; see FIGS. 1(C), 1(D), and 2(F)). If it is desired to retrieve the hybridizing genomic DNA containing vectors, for example plasmids, in their entirety, second strand synthesis may be performed with a strand displacing polymerase (eg. Klenow fragment of DNA polymerase I or φ29). Alternative methods include the use of an RNase in conjunction with a DNA polymerase (e.g. DNA polymerase I) to synthesize the second strand. During synthesis of the second strand, the 5′ end of the second strand is digested by exonucleolytic activity and the ends of the second strand are ligated together using a DNA ligase (eg. T4 DNA ligase) completing the circle. Single stranded plasmids on which hybridization and second strand DNA synthesis did not occur are removed, for example but not limited to, DNA nucleases such as S1 nuclease, or Mung bean nuclease, as described in the art. The remaining double stranded molecules including plasmids and re-hybridized cDNA can be transformed into any host cell.

A DNA polymerase that possesses 3′ exonucleolytic activity may be used if poly (A) or poly (T) tails, or non-hybridizing primer DNA, is present on a cDNA primer (for example if produced using the method of FIG. 7 as described below), or a DNA polymerase that can displace DNA strands may also be used. It is also contemplated that non-displacing polymerases may also be used as required (e.g. see FIGS. 2(C) to (F)). To reduce mispriming, second strand synthesis may take place at elevated temperatures using Taq polymerase. Many genes contain introns that are not typically found in cDNA, and these genomic regions may form loops in the hybridizing structures (30; in FIGS. 1(B), 1(D), 2(C), 2(D) and 2(F)). Therefore, in order to retrieve the hybridizing genomic DNA containing vectors in their entirety, second strand synthesis would be performed with a DNA polymerase such as DNA polymerase I. Following synthesis, the vector is reconstituted, or if required, the ends of the vector can be ligated together using a DNA ligase (for example but not limited to T4 DNA ligase). Single stranded vectors on which hybridization and second strand DNA synthesis did not occur, or any single stranded cDNA molecules still present, are also removed by, for example but not limited to, S1 nuclease, or mung bean nuclease treatment. The remaining double stranded molecules can be transformed into any desired host, for example, but not limited to, yeast or plant cells. Preferably the vectors are introduced into bacteria for further manipulation, such as, but not limited to E. coli using electroporation or chemical methods as known to one of skill in the art. Transformed cells containing the vectors are identified by using standard procedures, such as selection using antibiotic resistance, or determination of a gene or a gene product encoded by the vector.

Second Strand Synthesis: in Presence of COP Oligo

When isolating gene flanking fragments, second strand synthesis is performed with a DNA polymerase, preferably a non-displacing DNA polymerase with no 5′ exonucleolytic activity but having 3′ exonucleolytic activity (eg. T4 DNA polymerase) to remove poly (A) or poly (T) tails, or non-hybridizing primer DNA (see FIG. 7). Since many genes contain introns that are not typically found in mRNA, these genomic regions will form loops (30) in the hybridizing, structures that will be removed by the S1 nuclease along with the single stranded region of the vector between the hybridizing cDNA and the COP oligo. Furthermore, double stranded regions (35) residing between single stranded sequences will also be lost (see FIG. 2(C)). This will result in a 5′ flanking fragment with the 5′ most exon attached or a 3′ flanking fragment with the 3′ most exon attached. If there are no introns in the gene, the attached sequences will correspond to the coding sequence of the hybridizing cDNA (for example FIGS. 1(A) and 1(C)). The double stranded vector is reconstituted, for example if the vector is a plasmid it is re-circularized by blunt end ligation or via the use of an adapter using a DNA ligase (eg. T4 DNA ligase). Alternatively, if the vector is a plasmid, the ends can be modified using a number of techniques known in the art to facilitate re-circularization, for example, addition of guanine residues can be done with an enzyme such a terminal deoxynucleotidetransferase as known to someone skilled in the art. Inclusion of a Bst XI site in the COP-1 oligonucleotide (the first nucleic acid, 25) allows cleavage of the plasmid leaving a DNA overhang composed of four cytosine residues that are complementary to the guanine tail thus facilitating ligation. The reconstituted vector can be used for transformation of any desired host cell, for example, which is not to be considered limiting, yeast cells, plant cells, or preferably bacteria such as E. coli, using any suitable method, for example electroporation or chemical methods. The transformed host cells containing the vectors may then be identified by selecting for the occurrence of a marker or other selection criteria, for example antibiotic resistance, as is well known in the art.

Therefore, the present invention provides a method to obtain one or more vectors containing genomic gene coding fragments, 5′ flanking fragments, 3′ flanking fragments, or a combination thereof, as outlined in FIGS. 1(A) to 1(D), comprising:

i) preparing a genomic library comprising genomic DNA fragments within a vector;

ii) producing a population of single stranded DNAs from the genomic library;

iii) hybridizing one or more nucleic acid primers to the population of single stranded DNAs;

iv) synthesizing a second strand from the single stranded DNAs using a nucleic acid polymerase, and the hybridized one or more nucleic acid primers, and producing a double stranded nucleic acid;

v) removing any single stranded nucleic acid; and

vi) reconstituting the vector.

The present invention also provides a method to obtain one or more vectors containing genomic gene coding fragments, enriched for 5′ flanking fragments, 3′ flanking fragments, or a combination thereof suitable for further characterization, as outlined in FIG. 2(A), comprising:

i) preparing a population of single stranded genomic DNA fragments;

ii) hybridizing one or more nucleic acid primers to the population of single stranded genomic DNA fragments;

iii) synthesizing a second strand from the single stranded genomic DNA fragments using a nucleic acid polymerase, and the hybridized one or more nucleic acid primers, and producing double stranded nucleic acid;

iv) removing single stranded nucleic acid from the double stranded nucleic acid, to produce a population of double stranded DNA; and

v) introducing the double stranded DNA into a vector and reconstituting the vector.

Furthermore, the present invention provides an alternate method, as outlined in FIG. 2(B) to obtain one or more vectors containing genomic gene coding fragments enriched for 5′ flanking fragments, 3′ flanking fragments, or a combination thereof comprising:

i) preparing a genomic library within a vector;

ii) producing a population of single stranded DNAs comprising genomic DNA fragments from the genomic library;

iii) hybridizing a first nucleic acid to the population of single stranded DNAs and linearizing the vector to produce a population of linearized vectors;

iv) hybridizing one or more second nucleic acid primers to the population of linearized vectors;

v) synthesizing a second strand from the linearized vectors using a nucleic acid polymerase and the one or more second nucleic acid primers, and producing a double stranded nucleic acid;

vi) removing single stranded nucleic acid; and

vii) reconstituting the vector.

The present invention also provides a method to obtain one or more vectors containing genomic gene coding fragments enriched for 5′ flanking fragments, 3′ flanking fragments or a combination thereof, as outlined in FIGS. 2(C) to 2(F), comprising:

i) preparing a genomic library within a vector;

ii) producing a population of single stranded DNAs comprising genomic DNA fragments from the genomic library;

iii) hybridizing a modified first nucleic acid and one or more second nucleic acid primers to the population of single stranded DNAs;

iv) synthesizing a second strand from the single stranded DNAs using a nucleic acid polymerase and the one or more second nucleic acid primers, and producing double stranded nucleic acid;

v) removing single stranded nucleic acid; and

vi) reconstituting the vector.

The methods as described allow for the construction of libraries enriched in genomic sequences containing gene coding fragments, 5′ flanking fragments, 3′ flanking fragments, or combinations thereof. Alternatively, an affinity pair may be used to obtain a population of a 5′ or 3′ ends suitable for use as nucleic acid primers useful for obtaining genomic libraries comprising gene coding fragments enriched with 5′ flanking fragments, 3′ flanking fragments, or a combination thereof, using variations of the methods described herein.

An example of an affinity pair, which is not to be construed as limiting in any manner, comprises biotin and streptavidin. If biotin is used, then biotinylated (tagged) nucleic acids, for example, cDNAs or mRNAs can be hybridized to the genomic DNA and the tagged nucleic acid/genomic DNA hybrid selected using the second member of the affinity pair, for example, magnetic streptavidin particles (GeneTrappero® system; GibcoBRL). In this method, a tagged specific nucleic acid is used to enrich for certain DNAs within a library. Another example of an affinity system, which is not to be considered as limiting in any manner, consists of gene specific primers covalently linked to a particle surface (for example, Andreadis, J. D. and Chrisey, L. A., Nucleic Acids Res., 2000, 28, e5: i-viii). Immobilized nucleic acids may be used in the present invention to enrich for hybridizing cDNA, RNA or genomic DNA. However, it is to be understood that any affinity pair may be used with the above, or the following methods of the present invention.

Affinity pairs (the use of which is described in more detail below with reference to FIGS. 2(A), and 3-7) may be used to enrich for nucleic acid primers comprising 5′ and 3′ ends. This is especially useful in organisms where sequences coding for structural genes only make up a small proportion of the genome (for example but not limited to, wheat, oat, canola, alfalfa, or maize) or for organisms where the construction of good quality cDNA libraries is difficult. This enrichment can greatly facilitate molecular analysis and accelerate genome or gene sequencing efforts. Furthermore, these enriched genomic fractions can be used directly or may be used in other methods of the present invention.

Populations of 5′ or 3′ cDNA ends can be obtained using any suitable method. For example, which are not to be considered limiting in any manner, several approaches are described below with reference to FIGS. 3 to 7, however, other methods or variations on these methods may also be used.

Producing Antisense cDNA 5′ Ends

With reference to FIG. 3, a method outlining the production of a sub-population of antisense cDNA 5′ ends enriched for fragments encoding the 5′ end of mRNAs is outlined. In this method, full-length mRNAs are tagged using any suitable method, preferably, the tag is a first member of an affinity pair. For example, the tag may comprise biotin, and the mRNA may be biotinylated (see for example, Carninci, P. and Hayashizaki, Y., Methods Enzymol., 1999, 303:19-44). However, other tags, and methods for tagging full-length mRNA may also be employed. The tagged mRNAs are then hybridized with small cDNA fragments (single stranded or denatured double stranded) with sizes preferably ranging from 50-300 bp and which have preferably been synthesized using random oligomers to reduce the number of poly (T) tails, and generated by restriction digest or other methods. Following hybridization the RNA/DNA hybrids are treated with a nuclease, which cleaves single stranded RNA, for example but not limited to RNase I. The 5′ end RNA/DNA hybrids are then captured using the second member of the affinity pair. Preferably the second member of the affinity pair is attached to a substrate that is adapted for purification. For example, if biotin is used and a first member of an affinity pair, the second member of the affinity pair may comprise streptavidin. An example of a substrate adapted for purification includes, but is not limited to magnetic particles, chromatographic media, wells of a microplate, or filter paper, each derivatized so that the second member of the affinity pair is attached to the substrate. For example, streptavidin may be attached to magnetic particles. Following binding of the tagged mRNA/DNA hybrid with the second member of the affinity pair, the residual nucleic acids may then be washed away, and the RNA of the RNA/DNA hybrid can be destroyed using any suitable method, for example, but not limited to enzymatic treatment using RNase H, or chemical hydrolyzation using sodium hydroxide as is well known in the art. The remaining DNA corresponds to small single stranded cDNA fragments complementary to the 5′ end of full-length mRNAs. These antisense 5′ cDNA ends may be used to enrich for coding and 5′ flanking regions as described with reference to FIGS. 1(A), 1(B), 2(A) to 2(E), of the present invention.

Alternatively, a first strand cDNA synthesis can be performed followed by biotinylation of the diol groups (Carninci, P. and Hayashizaki, Y., Methods Enzymol., 1999, 303:19-44). The RNA/DNA hybrids can be treated with RNaseH to create nicks in the RNA along with a DNA nuclease to cleave the DNA strand opposite the RNA nicks, this will fragment the cDNAs and the 5′ RNA/DNA hybrids can be selected using any suitable methods, for example with magnetic streptavidin particles as outlined above.

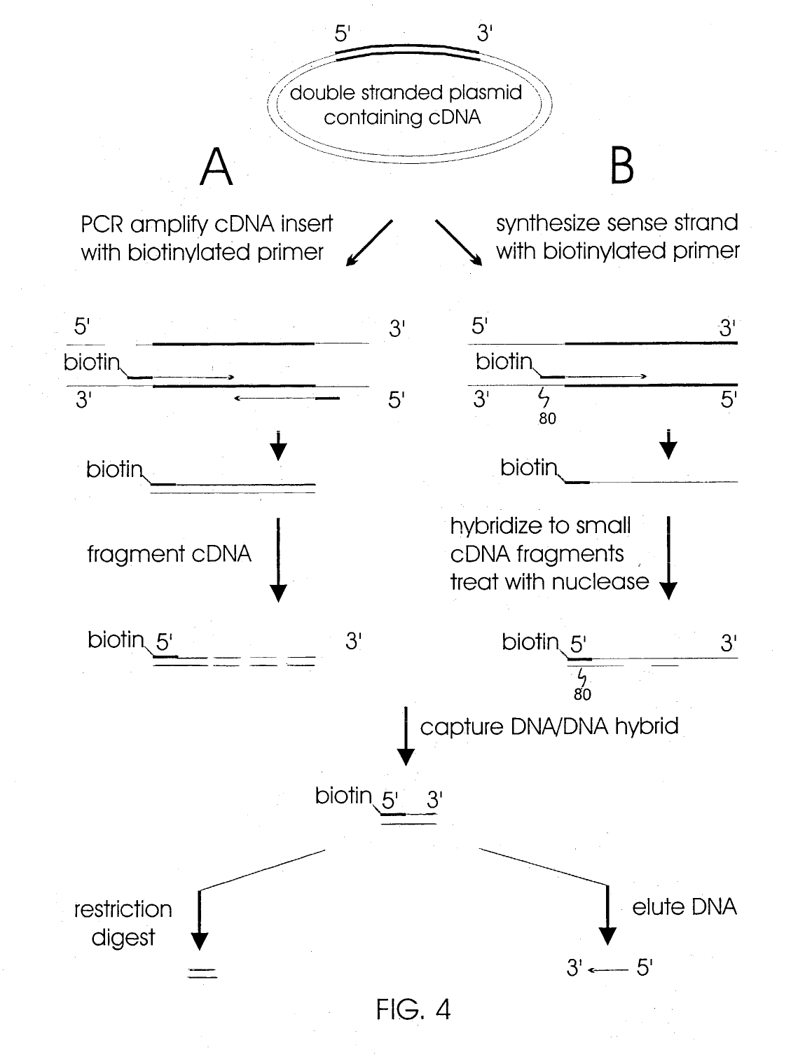

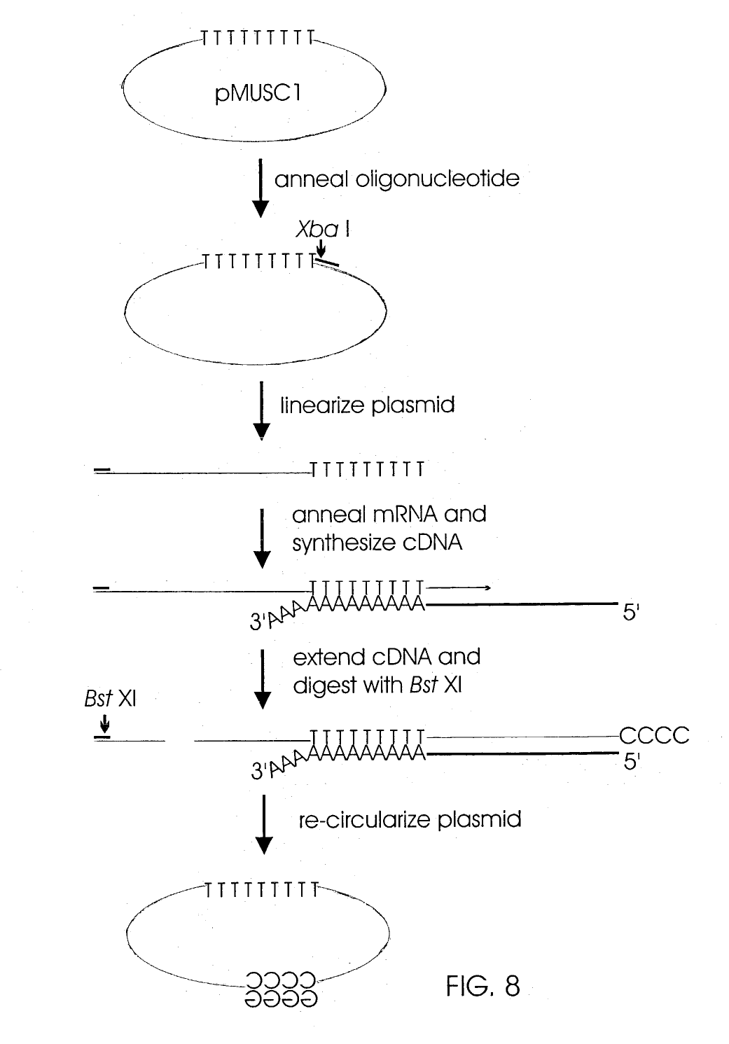

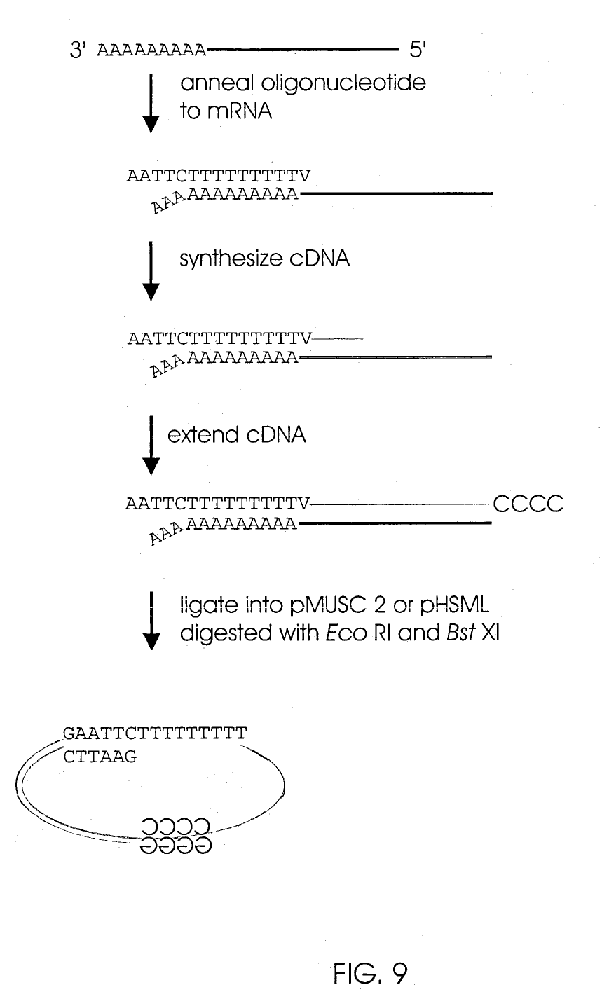

With reference to FIGS. 4(A) and (B), the present invention provides an alternate method for obtaining antisense 5′ cDNA ends corresponding to the 5′ end of mRNAs. In this method the initial template is preferably a directional full-length cDNA library obtained by methods known in the art, for example but not limited to the method outlined in FIGS. 8 and 9 (described in more detail below), in Carninci and Hayashizaki (Methods Enzymol., 1999, 303:19-44) or in Schmidt and Mueller (Nucleic Acids Res., 1999, 27:i-iv). In FIG. 4(A) PCR is performed with a first, tagged, primer. The tag of the primer comprises a first member of an affinity pair, for example but not limited to biotin. The first, tagged, primer is complementary to vector sequence upstream of the cDNA insert, and is used in combination with a second primer complementary to the vector region downstream of the insert. In this manner only the sense strands are tagged. Following PCR, the cDNA products are fragmented by restriction digest or other methods known in the art and the tagged DNA/DNA hybrids are captured using the second member of the affinity pair, for example but not limited to streptavidin, that is preferably attached to a substrate adapted for purification, for example but not limited to magnetic particles. The bound single-stranded antisense 5′ cDNA end is released from the captured hybrid, or the bound DNA is digested to release a double-stranded cDNA ends that can then be denatured before hybridization. Alternatively, biotin (tag) can be added to the cDNA via the ligation of an adaptor that is biotinylated (tagged).

With reference to FIG. 4(B), there is provided another alternate method for producing a population of antisense 5′ cDNA ends. In this method, vectors are preferably linearized at the 3′ end of the cDNA insert by restriction digest. The sense strand is synthesized by a DNA polymerase using a first tagged primer (tagged with a first member of an affinity pair), as described above, which hybridizes to the plasmid at the 3′ end of the antisense strand of the cDNA insert. This can be performed by asymmetric PCR using Taq polymerase for example, but can also be achieved by other methods known to one skilled in the art. The tagged cDNA is then hybridized with small cDNA fragments (single stranded or denatured double stranded) with sizes ranging from 50-300 bp preferably, generated by restriction digest or other method as described above, and then treated with a DNA nuclease such as S1 nuclease which cleaves single stranded DNA regions. Preferably, the cDNA fragments that are used to hybridize the tagged cDNA are cloned into, and prepared from, a vector comprising the same sequence as that of the insert, so that a complementary strand to the tagged primer (80) is available to ensure that the tagged primer is not digested. The tagged DNA/DNA hybrids are captured using the second member of the affinity pair, for example, which is not to be construed as limiting in any manner, if the tag is biotin, then the hybrids may be captured using magnetic streptavidin particles. The small single stranded antisense cDNA fragments corresponding to the 5′ ends of mRNA are released by denaturation or the bound DNA is digested to release a double-stranded cDNA ends that can then be denatured before hybridization. These antisense 5′ cDNA ends may be used to enrich for coding sequences and 5′ flanking sequences using the methods described in reference to FIGS. 1(A), 1(B), and 2(A) to (E).

The small single stranded cDNA fragments obtained by the methods outlined in FIGS. 4(A) and 4(B) can be used to hybridize to vectors containing genomic DNA and serve as primers for second strand synthesis as shown in FIGS. 1(A), 1(B), and 2(A) to (E). Since the last nucleotides of the 3′ end of the small cDNA fragments may comprise an oligonucleotide sequence corresponding to the flanking sequence of the vector used to synthesize the tagged cDNA (80; FIG. 4), this short sequence corresponding to the vector will not hybridize to genomic DNA and will be removed by the T4 DNA polymerase during second strand synthesis.

Producing Sense 5′ cDNA Ends

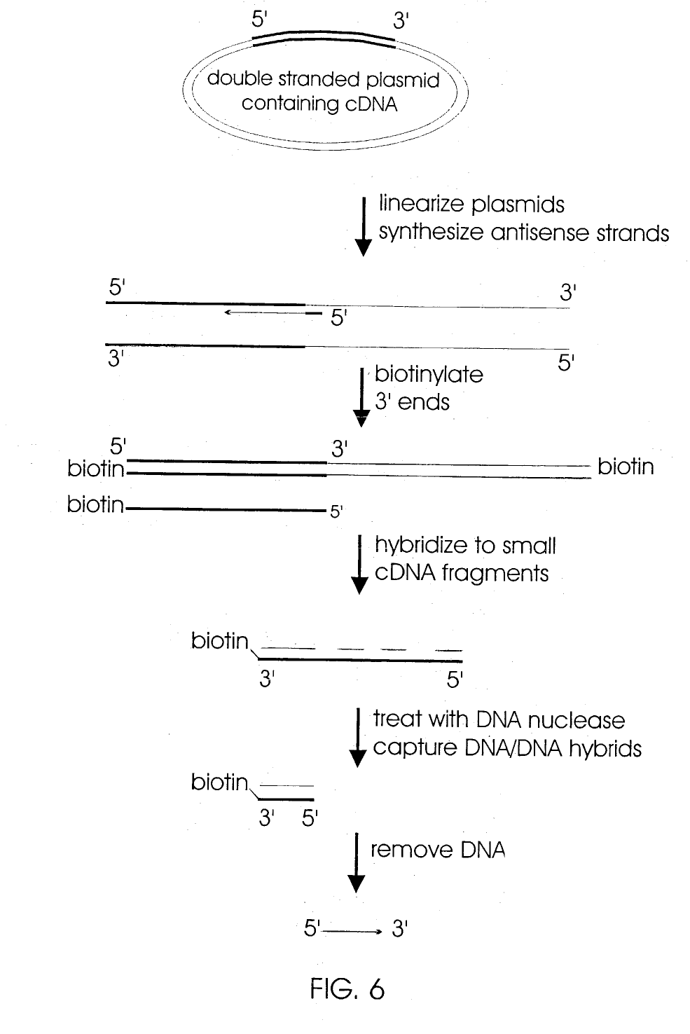

FIG. 6, shows an outline of a method for producing sense 5′ cDNA ends. In this method the initial template for the hybridization to small cDNA fragments is preferably a directional full-length cDNA library obtained by methods known in the art, for example, but not limited to the method described with reference to FIGS. 8 and 9 (see below), in Carninci and Hayashizaki (Methods Enzymol., 1999, 303:19-44) or in Schmidt and Mueller (Nucleic Acids Res., 1999, 27:i-iv). Preferably full-length mRNA is used to prepare the full-length cDNA library so that the 5′ ends of the cDNA correspond to the end of the mRNA. The vectors are preferably linearized at the 5′ end of the cDNA insert by restriction digest. The antisense strand is synthesized by a DNA polymerase using a primer that hybridizes to the vector at the 3′ end of the sense strand of the cDNA insert. This can be performed by asymmetric PCR using Taq polymerase for example, but can also be achieved by other methods known to one skilled in the art. The 3′ ends of all linear cDNAs are then tagged using a first member of an affinity pair, for example but not limited to biotin. If biotin is used, the 3′ ends may be chemically biotinylated. This step also tags the vector, therefore, the tagged vector may be removed by restriction enzyme digestion or by size separation after capture. The tagged cDNAs are then hybridized with small cDNA fragments (single stranded or denatured double stranded) preferably with sizes ranging from 50-300 bp, generated by restriction digest or other methods as described above and then treated with a DNA nuclease such as S1 nuclease which cleaves single stranded DNA regions. Preferably, the cDNA fragments that are used to hybridize the tagged cDNA are cloned into, and prepared from, a vector comprising the same sequence as that of the insert, so that a complementary strand to the tagged primer (80) is available to ensure that the tagged primer is not digested. The tagged DNA/DNA hybrids are captured using the second member of the affinity pair, as described above, for example using by magnetic streptavidin particles. The small single stranded cDNA fragments corresponding to the sense 5′ ends of mRNA are released by denaturation or the bound DNA is digested to release a double-stranded cDNA ends that can then be denatured before hybridization. These small fragments will serve to enrich for coding sequences and 3′ flanking sequences.

Producing Sense 3′ cDNA Ends

The above methods pertain to enriching for sub-populations of sense and antisense cDNA 5′ ends which can be used to isolate coding, 5′ flanking fragments as in FIGS. 1(A), 1(B), and 2(A) to (E), or other desired fragments. It is also possible to enrich for sub-populations of sense cDNA 3′ ends that correspond to the opposite strand of the template nucleic acid. These sub-populations of sense cDNA 3′ ends can be used to isolate coding sequences and 3′ flanking regions. An example of a method used to generate a sub-population of sense cDNA 3′ ends is shown with reference to FIGS. 5(A) and (B).

With reference to FIG. 5(A) there is shown an outline of a method for preparing sense 3′ ends that includes preparing a directional full-length cDNA library obtained by methods known in the art, for example but not limited to the method outlined in FIGS. 8 and 9 (described below), in Carninci and Hayashizaki (Methods Enzymol., 1999, 303:19-44), or in Schmidt and Mueller (Nucleic Acids Res., 1999, 27:i-iv). PCR is performed using a tagged first primer (see above) that is complementary to a vector sequence downstream of the cDNA insert, and a second primer complementary to the vector region upstream of the insert. This amplification therefore produces tagged antisense strands. Following PCR, the cDNA products are fragmented by restriction digest or other methods known in the art, and the tagged DNA/DNA hybrids are captured using the second member of the affinity pair attached to a suitable substrate, for example which is not to be considered limiting, if biotin is the first member of the affinity pair (i.e. the tag) then streptavidin, the second member of the affinity pair, may be attached to magnetic particles. The captured hybrid is then denatured and the sense 3′cDNA end recovered or the bound DNA is digested to release a double-stranded cDNA ends that can then be denatured before hybridization. Alternatively, biotin (tag) can be added to the cDNA via the ligation of an adaptor that is biotinylated (tagged).

With reference to FIG. 5(B), there is described an alternate method for the production of sense 3′ cDNA ends. In this method, vectors comprising full-length eDNA are preferably linearized at the 5′ end of the cDNA insert by restriction digest. The antisense strands are synthesized by a DNA polymerase using a biotinylated primer that hybridizes to the vector at the 3′ end of the sense strand of the cDNA insert. This can be performed by asymmetric PCR using Taq polymerase for example, but can also be achieved by other methods known to one skilled in the art. Alternatively, the antisense strands could be synthesized from plasmids linearized at the 3′ end of the cDNA inserts and these could then be tagged, for example biotinylated by chemical means. In either case, the tagged cDNAs are then hybridized with small cDNA fragments (single stranded or denatured double stranded) with sizes preferably ranging from 50-300 bp, generated by restriction digest or other methods as described above and then treated with a DNA nuclease such as S1 nuclease which cleaves single stranded DNA regions. Preferably, the cDNA fragments that are used to hybridize the tagged cDNA are cloned into, and prepared from, a vector comprising the same sequence as that of the insert, so that a complementary strand to the tagged primer (80) is available to ensure that the tagged primer is not digested. The tagged DNA/DNA hybrids are captured using the second member of the affinity tag, attached to a suitable substrate adapted for purification, for example magnetic streptavidin particles also as described above. The small sense cDNA fragments corresponding to the 3′ ends of mRNA are released by denaturation or the bound DNA is digested to release a double-stranded cDNA ends that can then be denatured before hybridization.

In FIGS. 7(A) and (B) there are shown alternate methods for the production of a population of sense cDNA 3′ ends. The initial template for the methods outlined in FIGS. 7(A) and (B) is mRNA which is first used for the synthesis of cDNA using an oligo d(T) primer tagged with a first member of an affinity primer. With reference to FIG. 7(A), following the synthesis of cDNA, a second cDNA strand is synthesized using the cDNA as a template and RNA fragments produced following an RNase H digestion as primers (e.g. Ausubel, F. M., et al., eds. Current Protocols in Molecular Biology, 2 vols., 1984, 1994, Wiley & Sons Inc.). The double stranded cDNA can be fragmented by restriction digest or other methods known in the art (for example, as described above). The tagged DNA/DNA hybrids are captured as outlined above, for example using magnetic streptavidin particles.

In FIG. 7(B) there is outlined a method, where after the first cDNA strand is synthesized, the mRNA is removed, and the cDNA is hybridized with small cDNA fragments (either single stranded or denatured double stranded cDNA) with sizes preferably ranging from about 50 to about 300 bp, generated by restriction digest or other methods as described above. The hybridized cDNA is then treated with a DNA nuclease such as S1 nuclease that cleaves single stranded DNA regions. The tagged DNA/DNA hybrids are captured by magnetic streptavidin particles. In the methods described with reference to both FIGS. 7(A) and 7(B), the sense cDNA 3′ ends are then eluted directly or the bound DNA can be digested to release double-stranded cDNA ends as described in FIGS. 4 and 5, if a suitable restriction site is present within the oligo d(T) primer used for cDNA synthesis, the resulting double-stranded ends are then denatured before hybridization.

Alternatively, instead of using biotin/streptavidin as the affinity pair to trap the cDNA, the synthesis of cDNA can be performed using oligo dT covalently attached to beads (for eg. Dynabeads Oligo d(T)₂₅, DYNAL). Following first strand cDNA synthesis and removal of mRNA, the cDNA is hybridized with cDNA fragments (single stranded or denatured double stranded) with sizes preferably ranging from 50-300 bp, and subsequently treated with DNA nuclease as described above. After removing unbound DNA, the small sense cDNA fragments corresponding to the 3′ end of mRNAs are denatured from the beads as described by the manufacturer and used to hybridize to plasmids containing genomic DNA as described in FIGS. 1(A), 1(B) and 2(A) to (E). Alternatively, the second strand of the bound cDNA can be synthesized and the cDNA fragmented as described in FIGS. 4(A) and 5(A), and following removal of unbound DNA, the sense cDNA 3′ ends can be released from the cDNA bound to oligo d(T).

Examples illustrated in FIGS. 5 and 7 describe several approaches to generate a sub-population of sense cDNA 3′ ends. However, it is to be understood that other methods may also be employed. Sense cDNA 3′ ends are useful for the isolation of gene coding sequences, including 3′ gene coding fragments and 3′ flanking fragments. However, sub-populations of antisense cDNA 3′ ends can also be useful to enrich for entire gene coding regions and 5′ flanking DNA. For example, antisense cDNA 3′ ends can be obtained using sense cDNA strands, synthesized from a vector preferably containing a directional cDNA library, for example by asymmetric PCR using a primer complementary to the vector sequence flanking the insert, and Taq polymerase, but can also be achieved by DNA synthesis from single stranded vector containing a directional cDNA library or by other methods known to one skilled in the art. Once sense cDNA fragments have been generated, their 3′ ends can be tagged, for example, biotinylated, and the tagged or biotinylated fragments can be used as disclosed in FIG. 6 to obtain antisense cDNA 3′ fragments corresponding to the 3′ ends of mRNAs and priming synthesis towards the 5′ of the genes. Alternatively, partial first strand cDNA synthesis from an mRNA template would also yield antisense cDNA 3′ ends.

The above examples provide different approaches to generate sub-populations of sense or antisense cDNA ends. These examples are not meant to be limiting. Alternative approaches, for example, cDNA ends can be amplified by PCR using sequence specific oligonucleotides or by using the oligonucleotide directly as a primer in the above protocol (FIGS. 1 and 2). Sub-populations from any nucleic acid sequence that has not been subcloned a priori or from any nucleic acid sequence, and subcloned in plasmids in a double stranded or single stranded circular or linear form, for example by using biotinylated primers in PCR reactions, direct chemical biotinylation or using other means of incorporating biotin as with the ligation of adapters, may also be used. These and other approaches will become obvious to someone skilled in the art. Similarly, although most examples provided use biotin, the proposed invention is not limited to this means of nucleic acid enrichment and encompasses other approaches such as using nucleic acids fixed to beads, as mentioned above. In the present invention, the methods using these sub-populations of cDNA ends to isolate gene coding and flanking DNA constitute one of the novel technologies proposed.

Procedure for Cloning Full-length cDNA

With reference to FIG. 8 there is provided an example of how cDNA can be obtained and directionally cloned using plasmid pMUSC 1 which is not meant to be limiting. pMUSC1 is isolated as a single stranded circular plasmid to which a single strand opener oligonucleotide, for example but not limited to SEQ ID NO: 7, is annealed. The resulting double stranded sequence is digested with Xba I that cleaves the plasmid right after the last dT of the 3′ end of the oligo dT stretch. Polyadenylated mRNA is annealed to the oligo dT stretch of the linear plasmid and used as a template to synthesize a first cDNA strand using reverse transcriptase (Ausubel, F. M., et al., eds. Current Protocols in Molecular Biology, 2 vols., 1984, 1994, Wiley & Sons Inc.). The first cDNA strand is being synthesized directly on to the 3′ end of the plasmid. The reaction is extended by addition of MnCl₂ which will generate a three to five dCs overhang at the 3′ end of the first strand (Schmidt, W. M. and Mueller, M. W., Nucleic Acids Res., 1999, 27:i-iv). A compatible 3′ dGs overhang can be produced on the plasmid by digesting the cDNA containing plasmid with Bst APT or Bst XI. The first strand is then ligated to one of the Bst sites and the product is used to transform a desired host, for example, E. coli cells.

FIG. 9 shows an outline of how cDNA can be obtained using the plasmids pHMSL1 or 2, or pMUSC2 that is not meant to be limiting. An oligo dT primer with a protruding 5′ sequence complementary to the overhang of a restriction site such as produced by Eco RI is annealed to mRNA and used to prime the synthesis of first cDNA strand (Ausubel, F. M., et al., eds. Current Protocols in Molecular Biology, 2 vols., 1984, 1994, Wiley & Sons Inc.). The first strand synthesis reaction is extended by the addition of MnCl₂. During the extension reaction, the enzyme reverse transcriptase adds an average of three to five dCs specifically to capped (full-length) rnRNA (Schmidt, W. M. and Mueller, M. W., Nucleic Acids Res., 1999, 27:i-iv). This first cDNA strand can be ligated directly into pHMSL1 or 2 digested with Eco RI and Bst API, or Bst XI, which produces a 3′ overhang of three dGs, or Bst XI, which generates a 3′ overhang of four dGs, or into a mixture of pHMSL1 or 2 digested with Eco RI and Bst API or Bst XI. This method ensures that the ligated first cDNA strand is cloned directionally into the vector. The cap-dependent generation of three to five dCs at the 3′ end of the first strand selects for full-length cDNAs since only those cDNAs can be ligated to the dG overhang of the vector. It is anticipated that in cases where the polyA tail is shorter than the stretch of dTs in the oligo dT primer that during the first strand synthesis step the 3′ end of the mRNA will also be extended. In that case cDNA could be digested with Eco RI to produce the compatible end required. Alternatively, double stranded versions of pMUSC2 or pHMSL can be digested with Eco RI and Bst API or Bst XI, and the Eco RI site is ligated to the dT stretch oligonucleotide (SEQ ID NO: 6) and annealed to mRNA. First stand synthesis and cap-dependent extension with dCs is carried out directly in the plasmid and the plasmid is re-circularized by ligating the compatible ends.

Alternate Method for the Production of cDNA 3′ Sense or Antisense, or 5′ Sense or Antisense Ends.

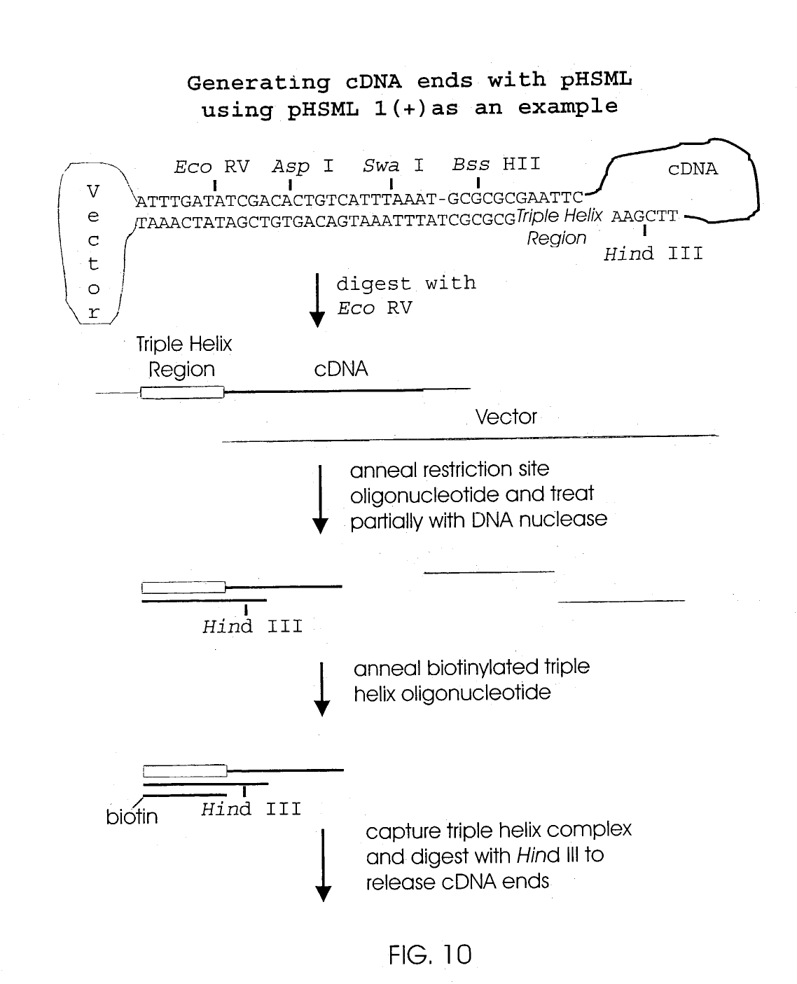

A method to produce single stranded cDNA ends using the plasmid pHMSL1 or 2 is illustrated in FIG. 10. cDNA, preferably full-length cDNA prepared for example as outlined above (see FIGS. 8 and 9), is cloned into pHMSL1 or 2 in both orientations with respect to the triple helix region (THR). The vector pHMSL1 is a pHelix (Roche Molecular Biochemicals) derivative with a Hind III site adjacent to the THR, while in pHMSL2, the multiple cloning site (MCS) is reversed such that the Eco RI site, which is on the opposite end of the MCS, is next to the THR. Both vectors, pHMSL1 and 2, are available as (+) or (−) vectors depending on which strand of the plasmid is generated as a single stranded circle by the f1 intergenic region. In FIG. 10, the isolation of single stranded cDNA ends using pHMSL plasmids is exemplified using pHMSL1 (+), but the same approach is applicable to all the pHMSL plasmids and the outlined method should not be considered limiting in any manner.

The cDNA is first cloned directionally (for example with Eco RI being at the 3′ end of the cDNA as described in FIG. 9) into pHMSL1 (+) and the plasmid is isolated as a single stranded circle. The cDNA sequence is therefore in the sense orientation with the Hind III site at the 5′ end. A single stranded cDNA end isolated from this vector will correspond to the 5′ end of the mRNA and will be in the sense orientation. The single stranded pHMSL vectors form a double-stranded region by hybridization of the stem-loop region between complementary sequences of the multiple cloning site located on either side of the insert (FIG. 10). This region can be digested using various restriction enzymes for example which is not to be considered limiting, Eco RV, Asp I, Swa I or Bss HII, to release the single-stranded insert. Following digestion, the single-stranded insert can be purified away from the remaining vector using the gel-free triple helix purification procedure (Roche Molecular Biochemicals). An oligonucleotide (rs oligo, SEQ ID NO: 8) complementary to the cDNA flanking region which contains the triple helix site and the flanking restriction sites is annealed to the single stranded insert. A single stranded DNA nuclease such as S1 nuclease is used in a partial digest to randomly hydrolyse the single stranded DNA. Different partial digests can be pooled. Alternatively, some restriction enzymes can digest single stranded DNA and these enzymes can also be used to reduce the size of the cDNA inserts. After the enzymatic digest, a triple helix oligonucleotide tagged with one member of an affinity pair, for example a biotinylated triple helix oligonucleotide (Roche Molecular Biochemicals), is annealed to the double stranded region consisting of the insert and the rs oligonucleotide. The resulting triple helix complex is captured using the second member of the affinity pair, for example, paramagnetic particles covered with streptavidin and separated from the remaining nucleic acids. The triple helix complex can then be released from the paramagnetic particles by raising the pH to 8.0 and briefly incubating in elevated temperature. The double stranded ends with the rs oligonucleotide are then digested with Hind III. After the digest, the pH is lowered again to promote formation of the triple helix complex that can then be captured and the single stranded cDNA ends are released and collected. Alternatively, the rs oligonucleotide itself can be tagged and used to capture the cDNA ends, both these and the captured triple helix structures can also be digested directly on the beads or other support matrix, to release the single stranded cDNA ends.

In order to obtain all four possible cDNA ends, the cDNA is cloned into four vectors, pHMSL1 (+) or pHMSL1 (−) for isolating sense and antisense single stranded cDNA ends corresponding to the 5′ end of the mRNA, and pHMSL2 (+) or pHMSL2 (−) for isolating sense and antisense single stranded cDNA ends corresponding to the 3′ end of the mRNA. Oligonucleotides (rs oligonucleotides) for the other pHMSL vectors may include: SEQ ID NO: 8 for pHMSL1 (+); SEQ ID NO: 9 for pHMSL1 (−); SEQ ID NO: 10 for pHMSL2 (+) and SEQ ID NO: 11 for pHMSL2 (−).

Sub-populations of small cDNA fragments may also be used for the construction of libraries which contain gene coding fragments, 3′ flanking fragments, 5′ flanking fragments, or a combination thereof, as shown in FIGS. 1 and 2. Since small cDNA fragments are used in the hybridization step this increases the probability that the final genomic DNA fragment will contain only a small region of coding sequence. This is particularly useful for genes that contain no introns or large first exons in the case of 5′ flanking sequences or large last exons in the case of the 3′ flanking sequences. This therefore represents a refinement over methods described above, with reference to FIGS. 1 and 2, since by using flanking primers found on the plasmid used for the library construction, it is possible to generate PSTs or TSTs on a scale comparable to that seen for ESTs, but with more of the partial sequence consisting of the flanking sequence. Furthermore, the small cDNA fragment used to prime the second strand synthesis of the genomic DNA serves to identify which gene the flanking sequence is associated with, can be easily sequenced through during characterization, and can also serve in large scale hybridization experiments such as genomic or flanking arrays to establish the expression profile associated with thousands of 5′ or 3′ flanking fragments simultaneously. Therefore by having the flanking fragments directly attached to the partial coding fragments which hybridize to the probes used in experiments such as flanking arrays, it is possible to analyze the expression profile associated with a flanking fragment, and to obtain the target sequence directly.

These methods are directed to using nucleic acid primers, for example but not limited to cDNA or mRNA to isolate gene coding and 5′ and 3′ flanking fragments making use of the fact that a cDNA or mRNA represents a copy of a gene's coding sequence.

The methods of the present invention allow for the construction of genomic DNA libraries which contain gene coding fragments, 5′ flanking fragments, 3′ flanking fragments, or a combination thereof. Such libraries can be used for generating PSTs or TSTs for use in DNA array analyzes as described above.

The above description is not intended to limit the claimed invention in any manner, furthermore, the discussed combination of features might not be absolutely necessary for the inventive solution.

The present invention will be further illustrated in the following examples. However it is to be understood that these examples are for illustrative purposes only, and should not be used to limit the scope of the present invention in any manner.

### EXAMPLES

### Example 1

Production of a Genomic DNA Library

Isolation of Genomic DNA.

Genomic DNA (and RNA) from whole Arabidopsis thaliana plants is isolated by the method Chang, S., et al., Plant Mol. Biol. Rep., 1993,11:113-116, with some modifications. Briefly, tissue is collected and frozen in liquid nitrogen. The tissue is ground using a mortar and pestle and transferred immediately into extraction buffer preheated to 65° C. For each gram of tissue, 7 ml extraction buffer is used. Following two chloroform extractions, 0.25 vol. of a 10 M LiCl solution is added to the aqueous phase. After an overnight incubation at 4° C., the precipitated RNA is pelleted by centrifugation and resuspended in DEPC-treated water. This RNA will be used for cDNA production. To the remaining supernatant, 1 vol. of isopropanol is added and the precipitated DNA is pelleted immediatelybycentrifugation. The DNA pellet is resuspended in TE buffer and re-precipitated with 0.3 M sodium acetate and ethanol. The DNA is recovered by centrifugation and resuspended in TE buffer (10 mM Tris/HCl pH 8, 1 mM EDTA) to a final concentration of 1 mg/ml.

Partial Restriction Digest and Size Selection of Genomic DNA.

One hundred μg of genomic DNA from Arabidopsis thaliana is incubated with 30 units of the restriction enzyme Alu I and the recommended enzyme buffer in a volume of 200 μl. The solution is incubated at 37° C. and 50 μl aliquots are removed after 5, 10, 15 and 30 minutes, to which 5 μl of 0.5 M EDTA are added to stop the reaction. The individual aliquots are checked by loading 5 μl on an agarose gel. The aliquots are then pooled and applied to a low-melting-point agarose gel. Four DNA fractions in the range of 2-3 kb, 3.5-4.5 kb, 4.5-6 kb and 6-8 kb are separately excised and extracted from the gel by agarase digestion as recommended by the manufacturer (Roche Molecular Biochemicals). The Alu I digested DNA fractions are then subjected to dATP labelling by incubating 1.5 μg of each DNA fraction with 300 μM dATP, 2 mM MgCl₂, 2.5 units Taq polymerase (Invitrogen) in 50 μl containing the manufacturer's reaction buffer for 30 min. at 68° C. The reaction is then digested with 20 μg proteinase K (Roche Molecular Biochemicals) according to the manufacturer's protocol. After phenol/chloroform extraction and ethanol precipitation, the DNA is resuspended in 20 μl TE.

Cloning of Size-selected dATP Labelled Genomic DNA.

Construction of pH-T1:

Vector pH-T 1 is obtained by inserting the adapter element NXT (SEQ ID NO: 13) into plasmids pHelix1 (+) and pHelix1 (−) (Roche Molecular Biochemicals) digested with Bam HI and Pst I. The adapter element NXT introduces two separate Xcm I sites which leave 3′ T-overhangs when pH-T1 is digested with Xcm I. The T-overhangs allow the cloning of the dATP labelled genomic DNA or of PCR products with dA overhangs.

Construction of pHSX-T1:

Vector pHSX-T1 is obtained by first inserting the adapter element ESX (SEQ ID NO: 14) into plasmids pHelix1 (+) and pHelix1 (−) (Roche Molecular Biochemicals) digested with Eco RI and Kpn I. The adapter element ESX introduces different Sfi I sites (Sfi I-A and Sfi I-B) which allow directional cloning of DNA inserts. The adapter element NXT (SEQ ID NO: 13) is then inserted in the Bam HI and Pst I sites to produce plasmid pHSX-T1. The complete multiple cloning site of pHSX-T1 is shown in SEQ ID NO: 15.

Construction of pHSacX-T1:

Vector pHSacX-T1 is obtained by cloning the Bacillus subtilis sacB gene for levansucrase (GenBank Accession no. X02730) into pHSX-T1. The sacB gene inhibits the growth of E. coli in the presence of sucrose. In this example, the sacB gene serves to select against bacteria containing plasmids where the sacB gene has not been removed. The sacB gene is isolated as a Pst I/Nde I fragment from plasmid pBB169 and is cloned as a blunt fragment into the blunted Sac I site of pHSX-T1 (+) and pHSX-T1 (−) to give pHSacX-T1 (+) and pHSacX-T1 (−) respectively.

Cloning of dATP Labelled Genomic DNA:

Five μl of the dATP labelled size-fractionated genomic DNA is ligated into 50 ng of pHSX-T1 (+) or pHSacX-T1 (+) in a 20 μl reaction volume. After an overnight incubation at 15° C., the ligation reaction is cleaned up using a PCR purification kit (Roche Molecular Biochemicals; manufacturer's protocol) resulting in 50 μl of purified ligation products. Two μl of purified ligation mix are used to transform 25 μl of electro-competent DH12S cells (Gibco/BRL) according to the manufacturer's protocol. An average of 2×10⁴ cells per transformation event are obtained. The presence of inserts is verified by DNA minipreps on selected colonies.

Preparation of a Single-stranded Genomic DNA Library.

This protocol is based on “Preparation of Single-Stranded Phagemid DNA” in: Promega Protocols and Applications Guide, 2nd Edition (1991) Promega Corporation. An overnight culture of E. coli cells (eg. DH5-FT; DH12S; JC236) containing the phagemid of interest is prepared by inoculating 2 ml TYP medium (16 g Bacto-tryptone, 16 g Bacto-yeast extract, 5 g NaCl, 2.5 g K₂HPO₄ per litre) with a single colony from a fresh bacterial plate and shaking the culture at 37° C. Five ml of TYP containing the appropriate antibiotics are then inoculated with 100 μl of the overnight culture. After shaking 30 min. at 37° C., 10 μl of helper phage (eg. M13K07) at a concentration of 10¹¹ pfu/ml is added and the culture is incubated at 37° C. in a shaker for 6 hr. The bacteria are pelleted by a 5 min. centrifugation at 5000×g and the supernatant is mixed with 0.25 vol. of 3.75 M ammonium acetate and 20% PEG 8000. After incubation for 15 min. on ice, the phage particles are collected by centrifugation at 12000×g for 15 min., resuspended in 400 μl TE and the DNA is extracted twice with phenol/chloroform and precipitated with salt and ethanol.

### Example 2

Production of cDNA Ends.

Cloning of cDNA Inserts in pH-T1 (+)

Arabidopsis thaliana cDNA clones corresponding to tubulin A4 (SEQ ID NO: 16; TUA4), metallothionein 2 (SEQ ID NO: 17; MT2), prohibitin 2 (SEQ ID NO: 18; phb2) and histone 4 (SEQ ID NO: 19; H4) are obtained and cloned into pH-T1 (+). Total RNA is isolated from Arabidopsis according to the method of Chang et al. (Plant Mol. Biol. Rep., 1993,11:113-116). Poly A+ mRNA is isolated using the polyA Spin mRNA isolation kit (New England Biolabs) following manufacturer's recommendation. The cDNA clones are obtained by RT-PCR using an anchored oligo dT primer and a gene specific primer SEQ ID NO: 20 for tubulin A, SEQ ID NO: 21 for metallothionein 2, SEQ ID NO: 22 for prohibitin 2 and SEQ ID NO: 23 for histone 4.

Production of 5′ cDNA Ends

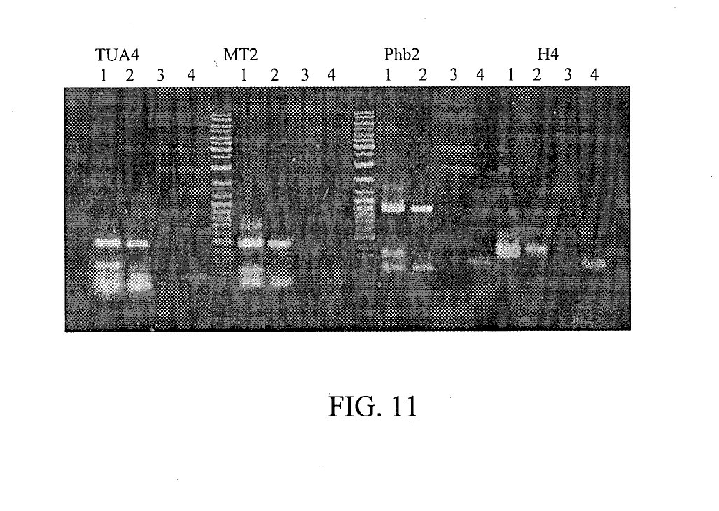

Biotinylated cDNA is produced by amplifying specific cDNA inserts subcloned in pH-T1 (+) in a PCR reaction containing a biotinylated T7 promoter-primer and a non-biotinylated T3 promoter-primer. The T3 and T7 promoter sequences flank the multiple cloning site of vector pH-T1 and PCR primers complementary to these promoter sequences can be used to amplify cloned inserts. Ten tg of biotinylated amplified cDNAs from tubulin A4 (SEQ ID NO: 16; TUA4), metallothionein 2 (SEQ ID NO: 17; MT2), prohibitin 2 (SEQ ID NO: 18; phb2) and histone 4 (SEQ ID NO: 19; H4) genes are produced by PCR using the biotinylated T7 promoter-primer and the T3 promoter-primer. The biotinylated PCR products are first digested with 10 units of Hpy CH4 III (TUA4, phb2, H4) or Ita I (MT), respectively, for 3 hr at 37° C. The biotinylated fragments are captured with 50 μl SA-PM beads (2 mg/ml streptavidin coated paramagnetic particles (Roche Molecular Biochemicals; manufacturer's protocol)) and the non-bound material is washed off. The bound DNA (representing the 5′ ends of the cDNA) is subjected to a second restriction enzyme digest that cleaves the bound DNA at the vector/insert junction by resuspending the beads in 50 μl restriction enzyme buffer solution and 10 units of Nla III for TUA4, phb2 and H4, or 10 units of Dde I for MT2 by incubating for 2 hr at 37° C. The eluate is collected and 5 μl containing the cDNA ends corresponding to the 5′ end of the mRNA are submitted to agarose gel-electrophoresis (FIG. 11).

Construction of pMUSC2

The Sfi adapter for pMUSC2 (SEQ ID NO: 2) is cloned into pHelix (Roche Molecular Biochemicals) between the Eco RI and Kpn I sites. The adapter provides two restriction sites for Sfi I (Sfi I-A and Sfi I-B) that allow directional cloning of cDNA. A Sac I site between the Sfi I sites is added to accommodate a stuffer fragment which facilitates the purification of the vector once digested with Sfi I and reduces background of undigested vector. This results in the intermediate plasmid pHSX. The Bst adapter for pMUSC2 (SEQ ID NO: 3) is cloned into pHSX between the Kpn I and Hind III sites. This adds restriction sites for NsiI, Bst API and Bst XI. Upon digestion, Bst API produces a 3′ overhang of three dGs, while digestion with Bst XI produces a 3′ overhang of four dGs. These sites provide compatible ends for the 3′ ends of first strand cDNAs (dC overhangs) produced by the method of Schrnidt, W. M. and Mueller, M. W., Nucleic Acids Res., 1999, 27:i-iv.

Production of pMUSC1

Vector pMUSC1 is obtained by digesting pMUSC2 with Sfi I and Xho I followed by the ligation of the oligo dT adapter (SEQ ID NO: 6) as a single strand. The resulting plasmid pMUSC1 has a stretch of 21 dT 5′ to 3′ between a Sfi I site and a Xba I site. This oligo dT stretch can be used as a priming site on mRNA in a single-stranded plasmid which has been linearized with Xba I by means of an oligonucleotide (SEQ ID NO: 7) that anneals at the Xba I site.

Construction of pHMSL

Construction of pHMSL is accomplished by first digesting pMUSC2 with Swa I and isolating the resulting fragment, which comprises all of the multiple cloning site, by the gel-free triple helix DNA purification method (Roche Molecular Biochemicals). The stem-loop adapter (SEQ ID NO: 4), whose sequence is entirely palindromic, is annealed to itself then ligated into the Swa I digested pMUSC2. The orientation of the adapter in the vector is verified by a double restriction digest. The resulting correct plasmid is then digested with Swa I that divides the stem-loop adapter in half with each half attached to one end of the plasmid. The multiple cloning site, which was rescued from the first digest, is then ligated into the Swa I digested vector thus restoring the Swa I site. The two halves of the stem-loop adapter are now flanking the multiple cloning sites. When the resulting plasmid, pHMSL, is isolated as a single-stranded circle, the two halves of the stem-loop adapter can now form a double helix spanning about 30 base pairs. This double-stranded region can now be digested with either Eco RV, Asp L Swa I or Bss HII, thus generating a single-stranded sequence comprising the DNA insert cloned into the vector. pHMSL1 is a pHelix (Roche Molecular Biochemicals) derivative with a Hind III site adjacent to the THR, while in pHMSL2, the multiple cloning site (MCS) is reversed such that the Eco RI site, which is on the opposite end of the MCS, is next to the THR. Both vectors, pHMSL1 and 2, are available as (+) or (−) vectors depending on which strand of the plasmid is generated as a single stranded circle by the f1 intergenic region.

### Example 3

Enrichment of Genomic DNA.

Several Arabidopsis thaliana cDNA clones, genomic fragments containing different genes, as well as fragments corresponding to the 5′ end of the mRNAs are isolated by PCR, examples of methods described herein to enrich genomic DNA include:

The cytochrome P450 genomic clone is obtained by PCR amplification from Arabidopsis genomic DNA using a forward primer (SEQ ID NO: 24) and a reverse primer (SEQ ID NO: 25) derived from GenBank sequence accession no. Z97337, and cloned into pHSacX-T1 (+). A 5′ end DNA fragment corresponding to the 5′ of the mRNA of the P450 gene is obtained by PCR using the forward primer (SEQ ID NO: 26) and the reverse primer (SEQ ID NO: 27) derived from GenBank sequence accession no. Z97337.

The interferon-like genomic clone is obtained by PCR amplification from Arabidopsis genomic DNA using the forward primer (SEQ ID NO: 29) and the reverse primer (SEQ ID NO: 30) derived from GenBank sequence accession no. AC007583, and cloned into pHSacX-T1 (+). A 5′ end DNA fragment corresponding to the 5′ of the mRNA of the interferon-like gene is obtained by PCR using the forward primer (SEQ ID NO: 31) and the reverse primer (SEQ ID NO: 32) also derived from GenBank sequence accession no. AC007583.

The RAD51 analog genomic clone is obtained by PCR amplification from Arabidopsis genomic DNA using the forward primer (SEQ ID NO: 33) and the reverse primer (SEQ ID NO: 34) derived from GenBank sequence accession no. AC007583, and cloned into pHSacX-T1 (+). A 5′ end DNA fragment corresponding to the 5′ of the mRNA of the RAD51 analog is obtained by PCR using the forward primer (SEQ ID NO: 35) and the reverse primer (SEQ ID NO: 36) also derived from GenBank sequence accession no. AC007583.

The TATA box binding protein association factor (bpaf) genomic clone is obtained by PCR amplification from Arabidopsis genomic DNA using the forward primer (SEQ ID NO: 37) and the reverse primer (SEQ ID NO: 38) derived from GenBank sequence accession no. AY050962, and cloned into pHSacX-T1 (+). A 5′ end DNA fragment corresponding to the 5′ of the mRNA of the TATA box bpaf is obtained by PCR using the forward primer (SEQ ID NO: 39) and the reverse primer (SEQ ID NO: 40) also derived from GenBank sequence accession no. AY050962.

The metallothionein 2 genomic clone is obtained by PCR amplification from Arabidopsis genomic DNA using the forward primer (SEQ ID NO: 41) and the reverse primer (SEQ ID NO: 42) derived from GenBank sequence accession no. AC011436, and cloned into pHSacX-T1 (+).

The prohibitin 2 genomic clone is obtained by PCR amplification from Arabidopsis genomic DNA using the forward primer (SEQ ID NO: 43) and the reverse primer (SEQ ID NO: 44) derived from GenBank sequence accession no. AC003027, and cloned into pHSacX-T1 (+).

The histone 4 genomic clone is obtained by PCR amplification from Arabidopsis genomic DNA using the forward primer (SEQ ID NO: 45) and the reverse primer (SEQ ID NO: 46) derived from GenBank sequence accession no. AF334729, and cloned into pHSacX-T1 (+).

The tubulin A4 genomic clone is obtained by PCR amplification from Arabidopsis genomic DNA using the forward primer (SEQ ID NO: 46) and the reverse primer (SEQ ID NO: 47) derived from GenBank sequence accession no. AC004809, and cloned into pHSacX-T1 (+).

Enrichment of Flanking Genomic DNA Sequences by Hybridization with a 5′ cDNA End and a Blocking Oligonucleotide

Enrichment of a Genomic Clone Containing Coding and Flanking Sequences of an Interferon-like Gene from Arabidopsis thaliana by First Linearizing the Plasmid Containing the Genomic Clone (FIG. 2B).

The interferon-like genomic clone is obtained by PCR amplification from Arabidopsis genomic DNA as describedabove (see interferon-like). In this example, isolation of the Arabidopsis thaliana interferon-like 5′ flanking and coding sequence is obtained from a mix of genomic Arabidopsis sequences containing the metallothionein 2, prohibitin 2, cytochrome P450, RAD51 analog and TATA box bpaf genes separately cloned into pHSacX-T1 (+) as described above.

One fig of each circular single-stranded plasmids containing genomic DNA inserts with the interferon-like, metallothionein 2, prohibitin 2, cytochrome P450, RAD5 1 analog and TATA box bpaf genes in both orientations is linearized by first annealing the blocking oligonucleotide COP-2 (SEQ iID NO: 28) to the single-stranded (ss) plasmid DNA. This is done by mixing 1 μg of each ssDNA and 100 pmoles blocking oligonucleotide COP-2 in a 20 μl volume containing the recommended buffer for the restriction enzyme Sfi I. The mix is placed on a block heater at 80° C., the heater is turned off and allowed to cool down to 50° C. Ten units of Sfi I enzyme (Roche Molecular Biochemicals) is added and the mix is incubated at 50° C. for 2 hr. After the digest is complete, the reactions containing the individual genomic clones are pooled and cleaned with a High Pure PCR Purification kit (Roche Molecular Biochemicals) which gives a final volume of 100 μl pooled DNA. Forty four μl of this solution are mixed with 0.5 μl of 20×SSC and 5 μl (1.5 μg) of denatured interferon-like 5′ end fragment. The hybridization mix is placed on a block heater at 80° C., the heater is turned off and allowed to cool down to 37° C. Second strand synthesis is performed by mixing 50 μl of the hybridization mix, 20 μl 5× T4 polymerase incubation buffer (Roche Molecular Biochemicals), 5 μl of 10 mM dNTPs, 1 μl of 5 μg/μl T4 gene 32 protein (Roche Molecular Biochemicals) and 2 μl (2 units) of T4 DNA polymerase (Roche Molecular Biochemicals), and incubating at 37° C. for 30 min. The reaction is cleaned up with a High Pure PCR Purification Kit that yields 100 μl of purified solution. Seventeen μl of this solution are mixed with 2 μl 10× T4 ligase buffer (New England Biolabs) and 1 μl T4 DNA ligase (New England Biolabs; 400 units/μl) and incubated at 15° C. for 16 hr. Two μl of the ligation mix are used to transform 100 μl competent DH5FT cells (GIBCO/BRL). Plasmid preparations resulting from the transformation were subjected to DNA sequencing.

SEQ ID NO: 47 is the nucleotide sequence of such a plasmid where the sacB gene and the 3′ portion of the interferon-like genomic clone were removed by the nuclease treatment during the experiment. The sequence data shows the remaining sequence of the blocking oligonucleotide after Sfi I digestion and nuclease blunting (nucleotide position 1 to 19), and the sequence of the new junction of the plasmid with the 5′ DNA tag that starts at nucleotide position 20.

Isolation of the 5′ Flanking Sequence from a Genomic Clone Containing Coding and Flanking Sequences of a Cytochrome P450 Gene from Arabidopsis thaliana.

The cytochrome P450 genomic clone is obtained by PCR amplification from Arabidopsis genomic DNA as described above (cytochrome P450). One hundred ng of the 5′ PCR double-stranded DNA fragment is denatured by boiling for 5 min. and then cooled for 5 min. on ice. Fifty ng of denatured 5′ DNA ends, 0.5 μg ssDNA of the P450 genomic clone in pHSacX-T1 (+), 200 pmoles of blocking oligonucleotide (SEQ ID NO: 28) and 8 μl 5× T4 polymerase buffer (Roche Molecular Biochemicals) are mixed in a volume of 22.5 μl. The hybridization mix is placed in a block heater at 70° C., the heater is turned off and allowed to cool down to 37° C. (ca. 1 hr). Second strand synthesis is performed by adding 1.5 μl of 10 mM dNTPs, 5 μl of 10 mM ATP, 1 μl of 5 μg/μl T4 gene 32 protein (Roche Molecular Biochemicals), 2 μl of 5× T4 polymerase buffer, 1 μl of T4 ligase (400 units, New England Biolabs), 2 μl T4 polymerase (2 units) and 15 μl water, to the hybridization mix. The mix is incubated for 2 hr at 37° C. then purified with a High Pure PCR Product Purification kit (Roche Molecular Biochemicals). A final volume of 100 μl is obtained after purification of which, 43 μl are subjected to restriction enzyme digest with 10 units Sfi I in a 50 μl volume for two hr at 50° C. The reaction is then placed at 30° C. and 5 units of mung bean nuclease (New England Biolabs) are added. After incubation for 30 min. at 30° C., the reaction is again cleaned up with the High Pure PCR Product Purification kit and 40 μl (80%) of the cleaned up blunt-ended DNA is ligated using 400 units of T4 ligase in a reaction volume of 50 μl. After incubation for 5 hr at room temperature, 2 μl of the ligation are used to transform 100 μl of competent DH5FT cells. Plasmid preparations resulting from the transformation were subjected to DNA sequencing and PCR analysis. DNA sequencing demonstrated that the 3′ end of the cytochrome P450 clone had been removed from the plasmids by nuclease treatment as expected.

Enrichment for Genomic Fragments Containing Coding.

Isolation of the RAD51 Analog from Arabidopsis thaliana from a Mixture of Genomic Arabidopsis Sequences Cloned as Described Above (RAD51) into pH-T1 (−) Using the PCR-amplified 5′ End from the RAD51 Gene as Primer.

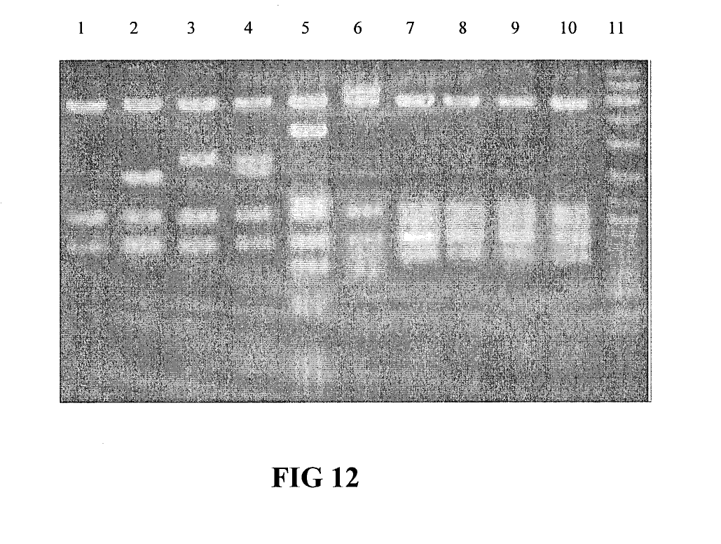

Circular single-stranded plasmids containing genomic DNA inserts of the Arabidopsis histone 4, interferon-like, prohibitin 2, RAD51 analog, cytochrome P450 and TATA box bpaf genes are annealed to the denatured 5′ ends of the RAD51 analog by mixing 1 μg of each single-stranded plasmid together and adding 1 μl of denatured 5′ ends and 0.5 μpl of 20×SSC in a volume of 35 μl. The mix is placed on a block heater at 80° C., the heater is turned off and allowed to cool down to 37° C. (ca. 1 hr). Second strand synthesis is performed by mixing 35 μl hybridization mix, 20 μl 5× T4 DNA polymerase incubation buffer (Roche Molecular Biochemicals), 5 μl of 10 mM dNTPs, 1 μl of 5 μg/μl T4 gene 32 protein (Roche Molecular Biochemicals), 10 μl of 10 mM ATP, 400 units of T4 ligase (New England Biolabs) and 2 units of T4 DNA polymerase (Roche Molecular Biochemicals) in a volume of 100 μl and incubating at 37° C. for 1 hr. The reaction is cleaned up with a High Pure PCR Purification kit (Roche Molecular Biochemicals) that yields 100 μl of purified solution. To 45 μl of this solution is added 5 μl of 10× mung bean nuclease incubation buffer and 1 μl mung bean nuclease (10 units, New England Biolabs). The mix is incubated at 30° C. for 20 min. and the reaction is cleaned up with the High Pure PCR Purification kit that yields 100 μl of purified solution. Two μl of the cleaned up solution are used to transform 100 μl competent DH5FT cells (GIBCO/BRL). Plasmid preparations resulting from the transformation were subjected to restriction digest analysis using Hinc II (FIG. 12).

### Example 4

DNA Arrays.

Recent technological developments now make it possible to monitor the expression of thousands of genes in a single experiment. DNA microarrays or macroarrays typically consist of DNA sequences (oligonucleotides, synthetic, cDNA or genomic DNA) that are first arrayed on a support (eg. microarrays/glass slides, macroarrays/filter membranes) and then hybridized with a labelled probe. Typically, the probe is labelled with a fluor (microarray), using radioactivity or other methods (macroarrays) normally used for Southern blot hybridizations as well known in the art.

The present invention allows for the use of DNA array technology for the large scale study of flanking genomic sequences such as regulatory regions, or sequences enriched for 5′ or 3′ flanking sequences, and hybridizing the arrayed DNA with a labelled cDNA probe. Alternatively, oligonucleotides could be derived from the sequences of FSTs, for example, and spotted or synthesized directly on glass slides, or used to generate sub fragments by PCR, before hybridization to the cDNA probe.

DNA Macroarrays:

Plasmids containing genomic DNA fragments enriched for 5′ flanking sequences are spotted on a Nylon membrane (Hybond N, Amersham Pharmacia) in a manner so that the location of each DNA fragment is known. It is preferred that the sequence information of the genomic fragments has been previously determined. The plasmids are spotted as purified plasmid DNA or as bacterial colonies containing these plasmids, as described (Clark et al., Methods Enzymol., 1999, 303:205-233). The arrayed DNA or colonies are fixed to the membrane and hybridized to the labelled cDNA probe as described by Sambrook et al., 1989, Molecular Cloning, 2^{nd} ed., Cold Spring Harbor Laboratory Press. Alternatively, the cloned inserts can be obtained (eg. by PCR) and spotted on the membrane.

Total RNA is isolated from Arabidopsis thaliana plants according to the method of Chang et al. (Plant Mol. Biol. Rep., 1993,11:113-116). Poly A+ mRNA is isolated using the polyA Spin mRNA isolation kit (New England Biolabs) following manufacturer's recommendation. Production of radio-labelled cDNA is achieved by reverse transcription using SuperScript II Rnase H⁻ reverse transcriptase (GIBCO BRL) following the manufacturer's instruction using 0.25 mM dCTP and 0.8 μM dCT³²P. Hybridization of the radioactive cDNA probes to complementary DNA present in the arrayed genomic DNA is determined using any desired method, for example, autoradiography, and reflects the gene expression levels associated with specific genomic DNA fragments found at the individual spots on the array.

Stock clones of the genomic DNA fragments identified in this analysis are identified and are further characterized. The corresponding regulatory regions for each of the identified genomic fragments may be characterized directly from the FST, or may be readily obtained by screening a genomic library using genomic DNA fragments identified from the micro array as a probe.

DNA Microarrays:

Although DNA chips can be produced using methods known in the art (for example, as described in http://www.affymetrix.com/technology/tech-probe.html) or by synthesizing oligonucleotides derived from the enriched genomic fragments obtained in Example 1, DNA fragments may also be directly spotted onto glass slides. The genomic DNA fragments enriched for 5′ flanking sequences are prepared as described above and arrayed on glass slides as described at the Ontario Cancer Institute website: http ://www.uhnres.utoronto.ca/services/microarray/products/index.html.

Fluorescently labelled cDNA probes were synthesized according to the methods outlined at http://www.uhnres.utoronto.ca/services/microarray/protocols/Pro_RT.html using poly A+ RNA isolated from the appropriate Arabidopsis thaliana tissue, using the above outlined protocols, and hybridized to the microarray using standard procedures: httt://www.uhnres.utoronto.ca/services/microarray/protocols/Pro_hybridization.html. This approach allows the use of two differentially labeled cDNA probes if desired (for eg. cDNA produced from two different sources of RNA labeled with Cyanine 3 and Cyanine 5 respectively). The hybridized slides are scanned using a SanArray 3000 Biochip Scanner (General Scanning Inc.).

All citations are herein incorporated by reference.

The present invention has been described with regard to preferred embodiments. However, it will be obvious to persons skilled in the art that a number of variations and modifications can be made without departing from the scope of the invention as described herein.

The ebodiments of the invention in which an exclusive property or privilege is claimed are defined as follows:

1. A method to obtain a vector comprising one or more gene coding fragments, flanking fragments, or a combination thereof, comprising:
i) providing a population of single stranded DNAs from a genomic library;
ii) hybridizing one or more nucleic acid primers to said population of single stranded DNAs;
iii) synthesizing a second strand from said single stranded DNAs using a nucleic acid polymerase, and said hybridized one or more nucleic acid primers, and producing a double stranded nucleic acid;
iv) removing single stranded nucleic acid; and
v) reconstituting said vector.

2. The method of claim 1, wherein in said step of hybridizing (step iii)), said one or more nucleic acid primer is a cDNA.

3. The method of claim 2, wherein said cDNA is a full-length cDNA, a 5′ cDNA end, a 3′ cDNA end, or a combination thereof.

4. The method of claim 2, wherein said cDNA is a 5′ cDNA end.

5. The method of claim 2, wherein said cDNA is a 3′ cDNA end.

6. The method of claim 1, wherein in said step of hybridizing (step iii)), said one or more nucleic acid primer is a full-length mRNA, or portion thereof, or an RNA fragment, or a combination thereof.

7. A method to obtain a vector comprising one or more gene coding fragments, flanking fragments, or a combination thereof, comprising:
i) providing a population of single stranded DNAs from a genomic library;
ii) hybridizing one or more nucleic acid primers to said population of single stranded DNAs, said one or more nucleic acid primers selected from the group consisting of 5′ cDNA ends, 3′ cDNA ends, RNA ends, and a combination thereof;
iii) synthesizing a second strand from said single stranded DNAs using a nucleic acid polymerase, and said hybridized one or more nucleic acid primers, and producing a double stranded nucleic acid;
iv) removing single stranded nucleic acid; and
v) reconstituting said vector.

8. A method to obtain a vector comprising genomic DNA, enriched for gene coding fragments, flanking fragments, or a combination thereof, comprising:
i) providing a population of single stranded genomic DNA fragments;
ii) hybridizing one or more nucleic acids to said population of single stranded genomic DNA fragments;
iii) synthesizing a second strand from the single stranded genomic DNA fragments using a nucleic acid polymerase, and said hybridized one or more nucleic acids, and producing double stranded nucleic acid;
iv) removing single stranded nucleic acid, to produce a population of double stranded DNA; and
v) introducing said population of double stranded DNA into said vector; and
vi) reconstituting said vector.

9. The method of claim 8, wherein in said step of hybridizing (step ii)), said one or more nucleic acid primer is a cDNA.

10. The method of claim 9, wherein said cDNA is a full-length cDNA, a 3′ cDNA end, a 5′ cDNA end, or a combination thereof.

11. The method of claim 9, wherein said cDNA is a 5′ cDNA end.

12. The method of claim 9, wherein said cDNA is, a 3′ cDNA end.

13. The method of claim 8, wherein in said step of hybridizing (step iii)), said one or more nucleic acid primer is a full-length mRNA, or portion thereof, or an RNA fragment, or a combination thereof.

14. A method to obtain a vector comprising genomic DNA enriched for gene coding fragments, flanking fragments, or a combination thereof comprising:
i) providing a population of single stranded DNAs comprising genomic DNA fragments from a genomic library;
ii) hybridizing a modified first nucleic acid, and one or more second nucleic acids to said population of single stranded DNA;
iii) synthesizing a second strand from said single stranded DNA using a nucleic acid polymerase and said one or more second nucleic acids as a primer for nucleic acid synthesis, and producing double stranded nucleic acid;
iv) removing single stranded nucleic acid; and
v) reconstituting said vector.

15. The method of claim 14, wherein in said step of hybridizing (step iii)), said one or more second nucleic acids is a cDNA.

16. The method of claim 15, wherein said cDNA is a full-length cDNA, a 3′ cDNA end, a 5′ cDNA end, or a combination thereof.

17. The method of claim 16, wherein said cDNA is a 5′ cDNA end.

18. The method of claim 16, wherein said cDNA is a 3′ cDNA end.

19. The method of claim 14, wherein in said step of hybridizing (step iii)), said one or more second nucleic acids is a full-length mRNA, or portion thereof, or an RNA fragment, or a combination thereof.

20. The method of claim 14, wherein in said step of hybridizing (step iii)), said modified first nucleic acid comprises an amine or thiol group at its 3′ end.

21. A method to obtain a vector comprising genomic DNA enriched for gene coding fragments, flanking fragments, or a combination thereof comprising:
i) providing a population of single stranded DNAs comprising genomic DNA fragments from a genomic library;
iii) hybridizing a first nucleic acid to said population of single stranded DNAs and linearizing said vector, to produce a population of linearized vectors;
iv) hybridizing one or more second nucleic acids to said population of linearized vectors;
v) synthesizing a second strand from said linearized plasmids using a nucleic acid polymerase and said one or more second nucleic acids as a primer for nucleic acid synthesis, and producing a double stranded nucleic acid;
vi) removing single stranded nucleic acid; and
vii) reconstituting said vector.

22. The method of claim 21, wherein in said step of hybridizing (step iii)), said one or more second nucleic acids is a cDNA.

23. The method of claim 22, wherein said cDNA is a full-length cDNA, a 3′ cDNA end, a 5′ cDNA end, or a combination thereof.

24. The method of claim 23, wherein said cDNA is a 5′ cDNA end.

25. The method of claim 23, wherein said cDNA is a 3′ cDNA end.

26. The method of claim 21, wherein in said step of hybridizing (step iii)), said one or more second nucleic acids is a full-length mRNA, or portion thereof, or an RNA fragment, or a combination thereof.

27. The method of claim 21, wherein in said step of hybridizing (step iii)), said modified first nucleic acid comprises an amine or thiol group at its 3′ end.

28. A promoter sequence tag (PST).

29. A 3′ sequence tag (TST).

30. A promoter sequence tag produced by the method of claim 4.

31. A promoter sequence tag produced by the method of claim 11.

32. A promoter sequence tag produced by the method of claim 17.

33. A promoter sequence tag produced by the method of claim 24.

34. An array comprising a plurality of 5′ flanking fragments, said 5′ flanking fragments attached to coding fragments.

35. An array comprising a plurality of 3′ flanking fragments, said 3′ flanking fragments attached to coding fragments.

36. An array comprising a plurality of 5′ flanking fragments and 3′ flanking fragments, said 5′ flanking fragments and 3′ flanking fragments attached to coding fragments.

37. An array comprising a plurality of 5′ flanking fragments, or a portion thereof.

38. An array comprising 3′ flanking fragments, or a portion thereof.

39. A method of preparing an array comprising a plurality of flanking fragments or portions thereof, with attached coding fragments, comprising, obtaining a population of DNA fragments produced by the method of claim 1; and applying said population of DNA fragments reconstituted vectors onto a support.

40. A method of preparing an array comprising a plurality of flanking fragments or portions thereof, with attached coding fragments, comprising, obtaining a population of DNA fragments produced by the method of claim 7; and applying said population of DNA fragments onto a supports.

41. A method of preparing an array comprising a plurality of flanking fragments, or portions thereof, with attached coding fragments, comprising, obtaining a population of DNA fragments produced by the method of claim8; and applying said population of DNA fragments onto a support.

42. A method of preparing an array comprising a plurality of flanking fragments, or portions thereof, with attached coding fragments, comprising, obtaining a population of DNA fragments produced by the method of claim 14; and applying said population of DNA fragments onto a support.

43. A method of preparing an array comprising a plurality of flanking fragments, or portions thereof, with attached coding fragments, comprising, obtaining a population of DNA fragments produced by the method of claim 21; and applying said population of DNA fragments onto a support.

44. A method to produce cDNA ends comprising,
1i) providing RNA;
ii) hybridizing cDNA fragments to mRNA to produce a DNA/RNA hybrid;
iii) producing DNA/RNA fragments from said DNA/RNA hybrid;
iv) selecting said DNA/RNA fragments that comprise said cDNA end; and
v) removing said RNA.

45. The method of claim 44, wherein said step of selecting (step iv)), involves the use of tagged mRNA.

46. The method of claim 44, wherein in said step of preparing (step i)), said mRNA is full-length mRNA.

47. A method of producing cDNA ends comprising,
i) providing DNA fragments;
ii) obtaining cDNA within a vector;
iii) hybridising cDNA to DNA fragments to produce a DNA/DNA hybrid, or a portion thereof;
iv) generating one or more DNA/DNA fragments from said DNA/DNA hybrid;
v) selecting said one or more DNA/DNA fragments that comprise said cDNA end; and
vi) recovering one member of said one or more DNA/DNA fragments.

48. The method of claim 47, wherein in said step of preparing (step i)), said cDNA is full-length cDNA.

49. A vector comprising a plurality of contiguous dT's adjacent a restriction site that is capable of cleaving the 3′ end of said contiguous dT's.

50. pMUSC1.

51. A vector comprising a nucleotide sequence that when digested with an appropriate restriction enzyme produces a 3′ overhang of contiguous dG's

52. pMUSC2.

53. A vector comprising a stem-loop adaptor sequence, said stem-loop adaptor sequence capable of forming a hybridized stem-loop structure when said vector is in single stranded form, said hybridized stem loop structure comprising one or more restriction sites.

54. pHMSL1.

55. pHMSL2.

56. An array comprising a plurality of 5′ flanking fragments, or a portion thereof, wherein said 5′ flanking fragments obtained from a plant.

57. An array comprising 3′ flanking fragments, or a portion thereof, wherein said 3′ flanking fragments obtained from plant.

58. An array comprising a plurality of 5′ flanking fragments, or a portion thereof, wherein said 5′ flanking fragments are not obtained from yeast.

59. An array comprising a plurality of 3′ flanking fragments, or a portion thereof, wherein said 3′ flanking fragments are not obtained from yeast.

60. An array comprising DNA fragments produced by the method of claim 1.

61. An array comprising DNA fragments produced by the method of claim 7.

62. An array comprising DNA fragments produced by the method of claim 8.

63. An array comprising DNA fragments produced by the method of claim 14.

64. An array comprising DNA fragments produced by the method of claim 21.


SEQUENCE LISTING (47 sequences)

SEQ ID NO: 1 - DNA, 27 bp, Artificial
  Artificial DNA Sequence
cccttgccatccccatggaagcttttt

SEQ ID NO: 2 - DNA, 49 bp, Artificial
  Artificial DNA Sequence
aattcggccattatggccgagctcggccgaggcggcctctcgagggtac

SEQ ID NO: 3 - DNA, 34 bp, Artificial
  Artificial DNA Sequence
gtaccatgcatcccttgccatccccatggaagct

SEQ ID NO: 4 - DNA, 38 bp, Artificial
  Artificial DNA Sequence
gatatcgacactgtcatttaaatgacagtgtcgatatc

SEQ ID NO: 5 - DNA, 39 bp, Artificial
  Artificial DNA Sequence
aattcgagagaattcactttttttttttttttttttttv

SEQ ID NO: 6 - DNA, 40 bp, Artificial
  Artificial DNA Sequence
tcgagttctagaaaaaaaaaaaaaaaaaaaaaggccataa

SEQ ID NO: 7 - DNA, 55 bp, Artificial
  Artificial DNA Sequence
aaaaagatcttgagctcccatggtacgtagggaacggtaggggtaccttcgaaag

SEQ ID NO: 8 - DNA, 34 bp, Artificial
  Artificial DNA Sequence
ccccatggaagctttttccttctttctctcttct

SEQ ID NO: 9 - DNA, 34 bp, Artificial
  Artificial DNA Sequence
agaagagagaaagaaggaaaaagcttccatgggg

SEQ ID NO: 10 - DNA, 30 bp, Artificial
  Artificial DNA Sequence
gagagaattctttccttctttctctcttct

SEQ ID NO: 11 - DNA, 30 bp, Artificial
  Artificial DNA Sequence
agaagagagaaagaaggaaagaattctctc

SEQ ID NO: 12 - DNA, 25 bp, Artificial
  Artificial DNA Sequence
gaagaagaagaagaagaagaagggg

SEQ ID NO: 13 - DNA, 50 bp, Artificial
  Artificial DNA Sequence
gatccatgcatctcttggcattctagagtaccaagagatgcatggctgca

SEQ ID NO: 14 - DNA, 50 bp, Artificial
  Artificial DNA Sequence
aattcggccattatggccgagctcggccgaggcggcctctcgagtggtac

SEQ ID NO: 15 - DNA, 234 bp, Artificial
  Artificial DNA Sequence
gcggccgcgtaatacgactcactatagggcgaatttaaattgcgcgcgaattcggccattatggccgagctcggccgaggcggcctctcgagtggtacccggggatccatgcatctcttggcattctagagtaccaagagatgcatggctgcaggcatgcaagctttttccttctttctctcttctgcgcgctatttaaattgttccctttagtgagggttaattggcggccgc

SEQ ID NO: 16 - DNA, 1628 bp, Arabidopsis thaliana
gagcgtcttcataaacgcccttcgtcttcttccctggaaatctagtttctttcttccacgaaaatgagagagtgcatttcgatccacattggtcaggctggtatccaggtcggaaatgcttgctgggagctgtactgtcttgaacatggcattcagcctgatggccagatgccgagtgacaagactgttggtggaggtgacgatgctttcaacaccttcttcagtgaaaccggtgcaggtaagcacgtcccacgtgctgtctttgttgatcttgagccaactgtgatcgacgaggtcaggactggtacttaccgtcagcttttccaccctgaacaactcatcagcggtaaagaagatgcagctaacaatttcgcccgtggtcattacaccattgggaaagagattgttgacctgtgcttagaccgtatcagaaagcttgctgataactgtactggtctccaaggattcctcgtcttcaacgctgttggtggagggactggacctggtcttggatctctcctccttgagagactttctgttgactacgggaaaaagtccaagttgggtttcacagtttacccatctccacaggtctctacctccgttgttgagccttacaacagtgtcctctccactcattctctcttgggacacactgatgtctccatcctcctcgacaatgaagctatctatgacatctgcagacgctccctaagcattgagagaccaacctacaccaacctcaaccgcctcgtctctcaggttatttcttccttgactgcttctctgaggtttgatggtgccttaaatgttgatgtcactgagttccaaaccaacttggtcccatacccaagaatccacttcatgcttcctcctatgccccagtcatctccgcagagaaagccttccatgagcaactctcagttgctgagatcacaaacagtgcttttgagccagcttccatgatggctaagtgtgacccacgtcacggaaagtacatggcttgctgtttgatgtaccgtggtgatgttgtccccaaggatgtaaacgcagctgttggcaccatcaagaccaagcgcactattcagtttgttgactggtgtcctactggattcaagtgtggtatcaactaccagccaccaacagttgttccaggaggtgatcttgctaaagtccagagagctgtttgcatgatctcaaactcgaccagtgttgctgaggtgttctcccgtattgatcacaagtttgatcttatgtacgccaaacgtgctttcgttcactggtatgtgggtgagggtatggaagaaggagaattctcagaggcacgtgaggatcttgcagcattggagaaggattatgaagaggtcggtgctgaaggtggtgacgatgaagatgatgaaggagaggaatactaagaagaatgtttctaaaaactttggatttgtgtgggtttctctataatctcgtcttgtgagaatgggctcaaaactcttgggagtctttaatcgtgtgtgttttaaaacctacttctctatcttttcgtagccatgttatctctctattatctatttcctttgtgtgttaaatcgtttctgcctttctggaaaaaaaaaaaaaaaaaaaaaaaa

SEQ ID NO: 17 - DNA, 553 bp, Arabidopsis thaliana
atcttctcagatctcttccaattttctagaaaaaacatgtcttgctgtggtggaagctgtggttgtggatctgcctgcaagtgcggcaatggttgcggaggttgcaaaaggtaccctgacttggagaacaccgccaccgagactcttgtcctcggtgttgctccggcgatgaactctcagtacgaggcttccggcgagactttcgttgccgagaatgatgcttgcaaatgcggatctgactgcaagtgcaacccttgtacctgcaaatgaagaacttcataaaccctaagtctgtaataaccctaatgttatgttaggtttgcttatatgtaataattggctgatttttccggtagttttgccggcgacgttggtctttctcttcttctgtgtgtgtttttatggtttggtcattaagatatctctgcaaagttttatctttgtgactttattaatcctaagactattatgggtttgtattaaagtttgcttctttcttgctcactacacaattaagattcaagcccaaaaaaaaaaaaaaaaaaaaaaaaaa

SEQ ID NO: 18 - DNA, 1270 bp, Arabidopsis thaliana
atctgaccgatttctcatttctaaaaccctaagcttcgccggcagctccgatcgtgatctttcataaatcctaccatcgccgtcgattaagagctgagctcaggaaatgagtttcaacaaagttcccaacattcctggagctcctgctctctctgcccttcttaaggtcagcgttattggtgggctcggtgtctatgcccttactwatagtctctacaatgtcgatggaggacaccgtgctgtcatgttcaaccgattaactggtatcaaggaaaaggtgtacccagaaggcacacattttatggtgccatggtttgaaaggccaatcayctatgacgttcgtgcacgaccttaccttgtagagagcaccactggtagtcatgatcttcagatggtgaaaattggattaagggttctcacacgtcccatgggtgaccgtttgcctcagatttaccgaacccttggcgagaactacagcgaaagggttcttccttccatcatccatgaaaccctaaaagcagtggtagctcagtacaatgcaagccagctcattacccagagagaagctgtgagcagagagatacgcaagattctgacagagcgggcttctaactttgacattgctcttgatgatgtctccatcacgactctgacattcggcaaagagttcaccgcagctatcgaggcaaaacaagttgctgctcaggaagctgaacgggctaagttcattgtggagaaagccgaacaagacaggagaagtgcggttatccgtgcgcagggagaagctaaaagtgctcagcttatcggacaagcaattgcgaacaatcaggcattcatcactctgagaaagattgaagctgcgagagagattgcacagaccattgcacaatctgctaacaaggtttacctgagctccaacgatctgttgcttaaccttcaagaaatgaacttggagcctaagaagtaaaagagaaggaacgaagtaagtttcttcatttcatattcatttcatttaaaagatctctccgccttggaagtaaaggatgggttggtgtttgaccaaatgaccctttctcgtgactgattctgaaaatagtctttaagaggacatattgagagagacgcagattggctttggtcagtattattcatcattttcaatgaaactttttgttgatattttcctgtgtttccccgggcaattgatgaagttgaatttaaaactcaataatattctatatgatctgagcaaaaaaaaaaaaaaaaaaaa

SEQ ID NO: 19 - DNA, 534 bp, Arabidopsis thaliana
aagaggaaagggaggaaaaggattgggaaagggaggagcgaagaggcacaggaaggttctgagggataacattcaaggaatcaccaagcctgctattcgtcgtcttgctcgtagaggtggtgtcaagcgtatcagtggtctcatctacgaagagaccagaggtgtcctcaagatcttcctcgagaatgttatccgtgacgccgttacctacactgagcacgccaggaggaagacggtgaccgccatggatgttgtctacgctttgaagaggcaaggtcgtactctctacggtttcggaggttaatccgatttgggggattagggtttatgcaagtttggggatttcttcttgtttctgagatccgtgttaaatggtttgaattagtacaaaagtaaattcaggagttagtttttgtttctcgtttttcgttgttattagccaatgtgatgtagtagttaattatcttttgcaagctttgtgctaagaaaatctaatgagaattatccatttgcctgcaaaaaaaaaaaaaaaaa

SEQ ID NO: 20 - DNA, 20 bp, Artificial
  Artificial DNA Sequence
gagcgtcttcataaacgccc

SEQ ID NO: 21 - DNA, 19 bp, Artificial
  Artificial DNA Sequence
atcttctcagatctcttcc

SEQ ID NO: 22 - DNA, 24 bp, Artificial
  Artificial DNA Sequence
tatctgaccgatttctcatttcta

SEQ ID NO: 23 - DNA, 23 bp, Artificial
  Artificial DNA Sequence
aagaggaaagggaggaaaaggat

SEQ ID NO: 24 - DNA, 24 bp, Artificial
  Artificial DNA Sequence
tttttgggaaggagtagggttgac

SEQ ID NO: 25 - DNA, 24 bp, Artificial
  Artificial DNA Sequence
cgtggctctgtggtttagttgttg

SEQ ID NO: 26 - DNA, 18 bp, Artificial
  Artificial DNA Sequence
aggccccaactttcatca

SEQ ID NO: 27 - DNA, 18 bp, Artificial
  Artificial DNA Sequence
gcggcgctcttcatctgg

SEQ ID NO: 28 - DNA, 33 bp, Artificial
  Artificial DNA Sequence
ttgcgcgcgaattcggccattatggccgagctc

SEQ ID NO: 29 - DNA, 19 bp, Artificial
  Artificial DNA Sequence
acggcgttccatcagacaa

SEQ ID NO: 30 - DNA, 23 bp, Artificial
  Artificial DNA Sequence
tggcagatacatcgtgggaaaac

SEQ ID NO: 31 - DNA, 24 bp, Artificial
  Artificial DNA Sequence
accccaacaccttaagacccattt

SEQ ID NO: 32 - DNA, 22 bp, Artificial
  Artificial DNA Sequence
ttcaaaacccggtaaaactcct

SEQ ID NO: 33 - DNA, 21 bp, Artificial
  Artificial DNA Sequence
ggccctatttgcggttttcag

SEQ ID NO: 34 - DNA, 23 bp, Artificial
  Artificial DNA Sequence
gctggctttctttgtgggtttct

SEQ ID NO: 35 - DNA, 22 bp, Artificial
  Artificial DNA Sequence
tcggtttggaatgtttgtgttg

SEQ ID NO: 36 - DNA, 24 bp, Artificial
  Artificial DNA Sequence
agtccgtctctttgtctccagtcg

SEQ ID NO: 37 - DNA, 24 bp, Artificial
  Artificial DNA Sequence
atggcgaaagaaattatggtgatg

SEQ ID NO: 38 - DNA, 24 bp, Artificial
  Artificial DNA Sequence
caggaagttggggaatagaatagc

SEQ ID NO: 39 - DNA, 19 bp, Artificial
  Artificial DNA Sequence
cagcctggggaagatgagc

SEQ ID NO: 40 - DNA, 24 bp, Artificial
  Artificial DNA Sequence
taatgggaagtaagaaataggtca

SEQ ID NO: 41 - DNA, 21 bp, Artificial
  Artificial DNA Sequence
actctcccctgaagcctcgta

SEQ ID NO: 42 - DNA, 23 bp, Artificial
  Artificial DNA Sequence
aaaactgccctgtgatgaatgga

SEQ ID NO: 43 - DNA, 20 bp, Artificial
  Artificial DNA Sequence
gtggggttaccgatgactcc

SEQ ID NO: 44 - DNA, 24 bp, Artificial
  Artificial DNA Sequence
ataaccgcacttctcctgtcttgt

SEQ ID NO: 45 - DNA, 23 bp, Artificial
  Artificial DNA Sequence
ctaccgctactcacctcgcactc

SEQ ID NO: 46 - DNA, 20 bp, Artificial
  Artificial DNA Sequence
caaacgccggtctcatcata

SEQ ID NO: 47 - DNA, 367 bp, Artificial
  Artificial DNA Sequence
ttgcgcgcgaattcggccaaaacccggtaaaactccttctccaaatgcttcatagctttccgcatcaaatcctgtcctttagcaagcttagcggagtccggattcatagcaacggtgctctgaatggcgtattgtaacttgccgagaacctcgaggaagcgtctgccttctaggcggttttgagcggagaagagggaagagaggctgcatgaagaagaagaagagtcgcagaggtgtggagtaacccattggcatatgaaggcgtcagctgcctccacactatcgccgattagagactcggagaagccctgttgntgttgcggcccgaccttagaggattttgataggtggtgggttaagagcacat